(19) United States
(12) Patent Application Publication
(10) Pub. No.: US 2009/0048191 A1
(43) Pub. Date: Feb. 19, 2009

(54) Therapeutic molecules for modulating stability of vegf

(75) Inventors: Elizabeth Piroska Rakoczy, Scarborough (AU); Robert Jeffery Marano, Willagee (AU)

Correspondence Address: SEED INTELLECTUAL PROPERTY LAW GROUP PLLC, 701 FIFTH AVE, SUITE 5400, SEATTLE, WA, 98104, US

(73) Assignee: Lions Eye Institute Limited, Nedlands (AU)

(21) Appl. No.: 11/547,339

(22) PCT Filed: Mar. 31, 2005
(86) PCT No.: PCT/AU2005/000469

§ 371 Date: Sep. 9, 2008

Related U.S. Application Data
(60) Provisional application No. 60/558,265, filed on Mar. 31, 2004.

(51) Int. Cl. A61K 31/711; C07H 21/04; C12N 15/87; C12N 5/06

(52) U.S. Cl. 514/44; 536/23.1; 435/455; 435/375

(57) ABSTRACT

This present invention discloses nucleic acid compositions and methods that are useful for treating ischemic conditions in animals, particularly in mammals such as humans. Specifically, the invention discloses nucleic acid molecules comprising or encoding a sequence that modulates the stability of a transcript from a vascular endothelial growth factor gene, as well as pharmaceutical compositions containing such molecules, which arm useful for modulating angiogenesis or vascularization, especially in methods for treating ischemic conditions.

### FIELD OF TEE INVENTION

This invention relates generally to the fields of ischemic diseases or conditions including cardiac, kidney and cerebral ischemias as well as ischemic conditions affecting the limbs and extremities. More particularly, it concerns nucleic acid compositions and methods that are useful for treating ischemic conditions in animals, particularly in mammals such as humans. Specifically, the invention provides nucleic acid molecules comprising or encoding a sequence that modulates the stability of a transcript from a vascular endothelial growth factor gene, as well as pharmaceutical compositions containing such molecules, which are useful for modulating angiogenesis or vascularization, especially in methods for treating ischemic conditions.

Bibliographic details of the publications numerically referred to in this specification are collected at the end of the description.

### BACKGROUND OF THE INVENTION

Vascular development is a fundamental requirement for all tissue growth and the absence of adequate tissue vascularization results in cells becoming deprived of oxygen and nutrients. This provides the stimulus for cells to produce angiogenic factors, which function to recruit new blood vessels into the deprived tissue. The most important of the angiogenic factors involved in new blood vessel formation is vascular endothelial growth factor (VEGF), which is highly regulated and consists of four isoforms resulting from alternate splicing of a single gene (1,2). A characteristic of all four isoforms is the presence of unusually long and GC rich 5′- and 3′-UTRs (1,2), which contain most of the important control and regulatory elements involved in the modulation of VEGF expression (reviews by 3,4). These elements include several internal ribosomal entry sites (IRES) (5,6) hypoxia response elements (HRE) (7) and a number of stabilizing and destabilizing sequences (8,9).

The importance of VEGF mediated vascularization in disease states makes it an attractive target for gene therapies. Several methods of downregulating VEGF for the treatment of tumors and ocular neovascularization are currently being explored (10-12). Additionally, the present inventors have previously described a sense oligonucleotide (DS-085) that targets the 5′-UTR of the VEGF gene and that has proven effective at down-regulating the transcription and subsequent translation of VEGF both in vitro and in vivo (13). Its mechanism of action has been postulated as being due to Hoogsteen hydrogen bonding within the major groove of the duplex DNA, causing polymerase arrest (14-18) and, similar to regulatory regions of other genes, this oligonucleotide is rich in GA purine residues (19,20).

### SUMMARY OF THE INVENTION

The present invention arises from the discovery of other control elements in the untranslated regions of the VEGF gene, which facilitate reduced gene expression. The inventors have found that oligonucleotides comprising one or more of these control elements or polynucleotides from which these elements are expressible, increase VEGF expression both in vitro and in vivo, enhance angiogenesis in vivo and are useful for treating or preventing ischemic conditions, as described hereafter.

Thus, in one aspect, the present invention provides an isolated nucleic acid molecule consisting essentially of at least one nucleotide sequence represented by the formula:

AₙWGGGGBₘ (I)

wherein W is A, T or U;
Aₙ is a sequence of n nucleotides wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

Suitably, each of Aₙ and Bₘ comprises the same or different nucleotides selected from A, T, U, G and C or derivatives or analogues thereof.

Advantageously, the nucleic acid molecule is capable of enhancing the expression of VEGF in a host cell that expresses VEGF. In illustrative embodiments of this type, the nucleic acid molecule is capable of enhancing the stability of a transcript from the VEGF gene in a host cell that expresses the transcript.

In some embodiments, the nucleic acid molecule comprises one or more tandem repeats of the nucleotide sequence represented by formula (I). In illustrative examples of this type, the nucleic acid molecule is represented by the formula:

[AₙWGGGGBₘ]ₚ (II)

wherein Aₙ, Bₘ and W are as defined for formula (I); and

p is an integer from 2 to about 20.

In some embodiments, the nucleotide sequence is selected from any one of AGGGG [SEQ D NO:1], TGGGG [SEQ ID NO:2] or UGGGG [SEQ ID NO:3]. In some embodiments, the isolated nucleic acid molecule consists essentially of one or more sequences selected from QGAGGAGGGGGAGGAG [SEQ ID NO:4] or AGGAAGAGGAGAGGGG [SEQ ID NO:5].

In some embodiments, the isolated nucleic acid molecule consists essentially of a nucleic acid sequence corresponding to an untranslated region of a VEGF transcript or portion thereof, which is at least 12, 13, 14, 15, 16, 17, 18, 19, 20, 25, 30 or 35 to about 100, 150, 200, 300, 400 or 500 nucleotides in length. Illustrative examples of such nucleic acid sequences are set forth in SEQ ID NO:6 and 7. The invention also contemplates nucleic acid molecules that comprise sequence regions that are about 99%, about 98%, about 97%, about 96%, about 95%, about 94%, about 93%, about 92%, about 91%, or even about 90% identical to a portion of one of those sequences, so long as the resulting degenerate nucleotide sequence retains sufficient homology to destabilize a transcript to which it is operably connected and to thereby increase the amount of VEGF in a host cell that expresses VEGF.

In some embodiments, the nucleic acid molecule is an oligonucleotide comprising at least one sequence represented by formula (I). Suitably, the oligonucleotide comprises at least 5, 6, 7, 8, 9 or 10 to about 15, 20, 25, 30, 35, 40, 50, 60, 70, 80, 90 or 100 nucleotides. Illustrative examples of such oligonucleotides are set forth in SEQ ID NO:9-36. Desirably, the oligonucleotide is nuclease resistant. In other embodiments, the nucleic acid molecule is a polynucleotide comprising a nucleotide sequence that encodes a transcript consisting essentially of at least one nucleotide sequence represented by formula (I). In still other embodiments, the nucleic acid molecule is a construct comprising such a polynucleotide operably connected to a promoter.

The nucleic acid molecules of the present invention may be prepared in a variety of compositions, and may also be formulated in appropriate pharmaceutical vehicles for administration to human or animal subjects to increase the expression of VEGF. Accordingly, in another aspect, the invention provides a pharmaceutical composition comprising one or more nucleic acid molecules as broadly described above, and optionally a pharmaceutically acceptable adjuvant, carrier or diluent. In yet another aspect, the invention provides methods for enhancing the expression of VEGF, comprising introducing a nucleic acid molecule that comprises at least one nucleotide sequence represented by formula (I) as defined above into a host cell that expresses VEGF.

The nucleic acid molecules of the present invention, and compositions comprising them increase angiogenesis and thus provide new and useful therapeutics or prophylactics for the treatment, prevention, control or amelioration of symptoms of a variety of conditions that will benefit from enhanced angiogenesis or vascularization. Suitably, these conditions are ischemic conditions, illustrative examples of which include cerebral ischemia; intestinal ischemia; spinal cord ischemia; cardiovascular ischemia; myocardial ischemia associated with myocardial infarction; myocardial ischemia associated with congestive heart failure (CHF), ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies. Accordingly, in still another aspect, the invention provides methods for increasing angiogenesis or vascularization in a tissue. These methods generally comprise contacting the tissue in which VEGF is expressible with a nucleic acid molecule comprising at least one nucleotide sequence represented by formula (I) as defined above or a pharmaceutical composition comprising such a nucleic acid molecule in an amount effective to increase the expression or amount of VEGF in the tissue. In some embodiments, the tissue is selected from brain tissue, intestinal tissue, spinal tissue, myocardial tissue, ocular tissue, liver tissue, kidney tissue, skin tissue, penile tissue, tissue containing a wound or graft tissue. In a related aspect, the invention provides methods for increasing angiogenesis or vascularization in a subject. These methods generally comprise administering to the subject an effective amount of a nucleic acid molecule comprising one or more nucleotide sequences each represented by formula (I) as defined above or a pharmaceutical composition comprising such a nucleic acid molecule to thereby increase angiogenesis or vascularization. In another related aspect, the invention provides methods for preventing or treating an ischemic condition or for reducing, preventing or treating ischemia-related tissue damage in a subject having or at risk of developing such condition or damage comprising administering to the subject an effective amount of a nucleic acid molecule comprising one or more nucleotide sequences each represented by formula (I) as defined above or a pharmaceutical composition comprising such a nucleic acid molecule to thereby treat the ischemic condition or to reduce the tissue damage.

In still other related aspects, the invention provides the use of a nucleic acid molecule that comprises one or more nucleotide sequences each represented by formula (I) as defined above in the manufacture of a medicament for increasing angiogenesis or vascularization, or for preventing or treating an ischemic condition, or for reducing, preventing or treating ischemia-related tissue damage.

The control elements defined herein are proposed to be RNA-destabilizing elements and hence reduce expression of a VEGF transcript by reducing the stability of that transcript. Accordingly, the inventors consider that the control elements defined herein can also be useful for destabilizing heterologous transcripts, which is desirable, for example, in applications that require transcripts with short half-lives (for example, transient reporter assays). Accordingly, a further aspect of the present invention provides a nucleic acid construct comprising a sequence that encodes a RNA destabilizing element and that is operably connected to a heterologous polynucleotide, wherein the RNA destabilizing element comprises at least one sequence represented by formula (I). In a related aspect, the invention provides methods for decreasing the stability of a transcript expressed from a polynucleotide. These methods generally comprise operably connecting a RNA destabilizing element to the polynucleotide, wherein the RNA destabilizing element comprises at least one sequence represented by formula (I) as defined above.

### BRIEF DESCRIPTION OF THE DRAWINGS

FIG. 1 is a graphical representation showing the concentration of VEGF protein in conditioned media of cultured cells. S3 had no significant effect on the expression of VEGF protein while S1 and S2 mediated a two fold increase. DS-085 decreased VEGF protein to less that 50% of the controls.

FIG. 2a is a photographic representation showing an agarose gel electropherogram of amplification products obtained from RT-PCR of VEGF mRNA extracted from cells incubated in the presence and absence of various oligonucleotides. β-actin is a stable expressing gene and was used to normalize the levels of VEGF mRNA.

FIG. 2b is a graphical representation showing a densitometric analysis of the electropherogram shown in FIG. 2a. mRNA levels were normalized against expression levels of β-actin and expressed as a percentage of the control sample.

FIG. 3 is a slit lamp photographic representation of rat eyes 7 days after injection of oligonucleotides into the anterior chamber. S1 and S2 mediated a strong neovascular response while the eye injected with S3 retained a normal phenotype.

FIG. 4 is a photographic representation showing that eyes injected with S3 remained free and clear of neovascularization using both CFP and FA (f and g respectively) with the injection site being clearly visible (a, white arrow). Seven days post injection with oligonucleotides S1 and S2 resulted in angiogenesis, which appeared as a distinct red band under CFP (c, yellow arrows). Subsequent FA revealed hyperfluorescence (d, yellow arrows) due to the leaky nature of the new blood vessels. Fourteen days post injection resulted in the development of intraretinal hemorrhage, which appears as black spots using CFP (e, red arrows) and hypo-fluorescence using FA (f, red arrows). After 21 days the intra-retinal hemorrhage had further developed (g, blue arrow) and appears as a large area of hypofluorescence (h, blue arrow).

FIG. 5a shows a sequence alignment of 5′-UTRs of several species. The homopurine region (boxed) was used to design oligonucleotides S1, S2 and S3. The ATG start codon is in bold type.

FIG. 5b shows the 5′-UTR and 3′-UTR of the human VEGF gene. Sites of destabilizing elements ((A/T)GGGG) are underlined.

TABLE A
Brief Description of the Sequences

| SEQUENCE ID | | |
|---|---|---|
| NUMBER | SEQUENCE | LENGTH |
| SEQ ID NO: 1 | Control element 1 | 5 nts |
| SEQ ID NO: 2 | Control element 2 | 5 nts |
| SEQ ID NO: 3 | Control element 3 | 5 nts |
| SEQ ID NO: 4 | Oligonucleotide S1 | 16 nts |
| SEQ ID NO: 5 | Oligonucleotide S2 | 16 nts |
| SEQ ID NO: 6 | VEGF 5′-UTR | 1039 nts |
| SEQ ID NO: 7 | VEGF 3′-UTR | 1923 nts |
| SEQ ID NO: 8 | Oligonucleotide S3 | 16 nts |
| SEQ ID NO: 9 | Oligonucleotide comprising control | 12 nts |
| | element | |
| SEQ ID NO: 10 | Oligonucleotide comprising control | 12 nts |
| | element | |
| SEQ ID NO: 11 | Oligonucleotide comprising control | 12 nts |
| | element | |
| SEQ ID NO: 12 | Oligonucleotide comprising control | 12 nts |
| | element | |
| SEQ ID NO: 13 | Oligonucleotide comprising control | 13 nts |
| | element | |
| SEQ ID NO: 14 | Oligonucleotide comprising control | 13 nts |
| | element | |
| SEQ ID NO: 15 | Oligonucleotide comprising control | 13 nts |
| | element | |
| SEQ ID NO: 16 | Oligonucleotide comprising control | 13 nts |
| | element | |
| SEQ ID NO: 17 | Oligonucleotide comprising control | 14 nts |
| | element | |
| SEQ ID NO: 18 | Oligonucleotide comprising control | 14 nts |
| | element | |
| SEQ ID NO: 19 | Oligonucleotide comprising control | 14 nts |
| | element | |
| SEQ ID NO: 20 | Oligonucleotide comprising control | 14 nts |
| | element | |
| SEQ ID NO: 21 | Oligonucleotide comprising control | 15 nts |
| | element | |
| SEQ ID NO: 22 | Oligonucleotide comprising control | 15 nts |
| | element | |
| SEQ ID NO: 23 | Oligonucleotide comprising control | 15 nts |
| | element | |
| SEQ ID NO: 24 | Oligonucleotide comprising control | 15 nts |
| | element | |
| SEQ ID NO: 25 | Oligonucleotide comprising control | 16 nts |
| | element | |
| SEQ ID NO: 26 | Oligonucleotide comprising control | 16 nts |
| | element | |
| SEQ ID NO: 27 | Oligonucleotide comprising control | 16 nts |
| | element | |
| SEQ ID NO: 28 | Oligonucleotide comprising control | 16 nts |
| | element | |
| SEQ ID NO: 29 | Oligonucleotide comprising control | 17 nts |
| | element | |
| SEQ ID NO: 30 | Oligonucleotide comprising control | 17 nts |
| | element | |
| SEQ ID NO: 31 | Oligonucleotide comprising control | 17 nts |
| | element | |
| SEQ ID NO: 32 | Oligonucleotide comprising control | 17 nts |
| | element | |
| SEQ ID NO: 33 | Oligonucleotide comprising control | 18 nts |
| | element | |
| SEQ ID NO: 34 | Oligonucleotide comprising control | 18 nts |
| | element | |
| SEQ ID NO: 35 | Oligonucleotide comprising control | 18 nts |
| | element | |
| SEQ ID NO: 36 | Oligonucleotide comprising control | 18 nts |
| | element | |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “5′-UTR” is meant the 5′ (upstream) untranslated region of a gene. Also used to refer to the DNA region encoding the 5′-UTR of the mRNA.

By “3′-UTR” is meant the region of a polynucleotide downstream of the termination codon of a protein-encoding region of that polynucleotide, which is not translated to produce protein.

By “about” is meant a quantity, level, value, dimension, size, or amount that varies by as much as 30%, preferably by as much as 20%, and more preferably by as much as 10% and even more preferably by as much as 9%. 8%, 7%, 6%, 5%, 4%, 3%, 2% or 1% to a reference quantity, level, value, dimension, size, or amount.

The phrases “consisting essentially of,” “consists essentially of” and the like refer to the components which are essential in order to obtain the advantages of the present invention and any other components present would not significantly change the properties related to the inventive concept.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The term “dendrimer” refers to branched macromolecules having polymeric “arms” that emanate from a core molecule.

By “effective amount”, in the context of treating or preventing a condition is meant the administration of that amount of active to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “endogenous” refers to a gene or nucleic acid sequence or segment that is normally found in a host cell or host organism.

By “expression vector” is meant a vector that permits the expression of a polynucleotide inside a cell. Expression of a polynucleotide includes transcriptional and/or post-transcriptional events

The term “gene” as used herein refers to any and all discrete coding regions of a host genome, or regions that code for a functional RNA only (for example, tRNA, rRNA, regulatory RNAs such as ribozymes etc) as well as associated non-coding regions and optionally regulatory regions. In certain embodiments, the term “gene” includes within its scope the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The gene sequences may be cDNA or genomic DNA or a fragment thereof. The gene may be introduced into an appropriate vector for extrachromosomal maintenance or for integration into the host.

An “ischemic condition” refers to a medical event which is pathological in origin, or to a surgical intervention which is imposed on a subject, wherein circulation to a region of the tissue is impeded or blocked, either temporarily, as in vasospasm or transient ischemic attach (TIA) in cerebral ischemia or permanently, as in thrombolic occlusion in cerebral ischemia. The affected region is deprived of oxygen and nutrients as a consequence of the ischemic event. This deprivation leads to the injuries of infarction or in the region affected. The present invention encompasses cerebral ischemia; intestinal ischemia; spinal cord ischemia; cardiovascular ischemia; ischemia associated with CHF, liver ischemia; kidney ischemia; dermal ischemia; vasoconstriction-induced tissue ischemia, such as a consequence of Raynaud's disorder, penile ischemia as a consequence of priapism; and ischemia associated with thromboembolytic disease; microvascular disease; such as for example diabetes and vasculitis; diabetic ulcers; gangrenous conditions; post-trauma syndrome; cardiac arrest resuscitation; and peripheral nerve damage and neuropathies; and other ischemias, including ischemia associated with ocular health concerns, such as for example, age-related macular degeneration (AMD). Ischemia occurs in the brain during, for example, a stroke, cardiac arrest, severe blood loss due to injury or internal hemorrhage and other similar conditions that disrupt normal blood flow. Ischemia occurs in myocardial tissue as a, result of, for example, atherosclerosis and CHF. It may also occur after a trauma to the tissue since the pressure caused by edema presses against and flattens the arteries and veins inside the tissue, thereby reducing their ability to carry blood through the tissue. Cerebral ischemia may also occur as a result of macro- or micro-emboli, such as may occur subsequent to cardiopulmonary bypass surgery. Age-related macular degeneration may be associated with oxidative damage to the retina as a result of an ischemic condition.

By “mRNA” is meant messenger RNA, which is a “transcript” produced in a cell using DNA as a template, which itself encodes a protein. mRNA is typically comprised of a 5′-UTR, a protein encoding (i.e., coding) region and a 3′-UTR. mRNA has a limited half-life in cells, which is determined, in part, by stability elements, particularly within the 3′-UTR but also in the 5′-UTR and protein encoding region.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 9 to 35 nucleotides, but the term can refer to molecules of any length, although the term “polynucleotide” or “nucleic acid” is typically used for large oligonucleotides.

The terms “operably connected,” “operably linked,” “in operable linkage,” “in operable connection” and the like are used herein to refer to the placement of a transcribable sequence under the regulatory control of a promoter, which controls the transcription and optionally translation of the sequence. In the construction of heterologous promoter/transcribable sequence combinations, it is generally desirable to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the desirable positioning of a regulatory sequence element with respect to a heterologous gene to be placed under its control is defined by the positioning of the element in its natural setting; i.e. the genes from which it is derived.

By “pharmaceutically acceptable carrier” is meant a solid or liquid filler, diluent or encapsulating substance that may be safely used in topical, local or systemic administration.

The terms “polynucleotide” and “nucleic acid” are synonymous and refer to a polymer having multiple nucleotide monomers. A nucleic acid can be single- or double-stranded, and can be DNA (cDNA or genomic), RNA, synthetic forms, and mixed polymers, and can also be chemically or biochemically modified or can contain non-natural or derivatized nucleotide bases. Such modifications include, for example, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al., supra, Science 254, 1497-1500, 1991). “Nucleic acid” or “polynucleotide” do not refer to any particular length of polymer and can, therefore, be of substantially any length, typically from about six (6) nucleotides to about 10⁹ nucleotides or larger. In the case of a double-stranded polymer, “nucleic acid” or “polynucleotide” can refer to either or both strands.

By “promoter” is meant a region of DNA, generally upstream (5′) of a coding region, which controls at least in part the initiation and level of transcription. Reference herein to a “promoter” is to be taken in its broadest context and includes the transcriptional regulatory sequences of a classical genomic gene, including a TATA box and CCAAT box sequences, as well as additional regulatory elements (i.e., activating sequences, enhancers and silencers) that alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Promoters according to the invention may contain additional specific regulatory elements, located more distal to the start site to further enhance expression in a cell, and/or to alter the timing or inducibility of expression of a structural gene to which it is operably connected. The term “promoter” also includes within its scope inducible, repressible and constitutive promoters as well as minimal promoters. Minimal promoters typically refer to minimal expression control elements that are capable of initiating transcription of a selected DNA sequence to which they are operably linked. In some examples, a minimal promoter is not capable of initiating transcription in the absence of additional regulatory elements (for example, enhancers or other cis-acting regulatory elements) above basal levels. A minimal promoter frequently consists of a TATA box or TATA-like box. Numerous minimal promoter sequences are known in the literature. For example, minimal promoters may be selected from a wide variety of known sequences, including promoter regions from fos, CMV, SV40 and IL-2, among many others. Illustrative examples are provided which use a minimal CMV promoter or a minimal IL2 gene promoter (−72 to +45 with respect to the start site; Siebenlist, 1986).

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, livestock animals (for example, sheep, cows, horses, donkeys, pigs), laboratory test animals (for example, rabbits, mice, rats, guinea pigs, hamsters), companion animals (for example, cats, dogs) and captive wild animals (for example, foxes, deer, dingoes). A preferred subject is a human in need of treatment or prophylaxis for an ischemic condition or ischemia related tissue damage. However, it will be understood that the aforementioned terms do not imply that symptoms are present.

The terms “treat,” “treating” and the like include both therapeutic and prophylactic treatment.

By “vector” is meant a vehicle for inserting a foreign DNA sequence into a host cell and/or amplifying the DNA sequence in cells that support replication of the vector. Most commonly a plasmid but can also be a phagemid, bacteriophage, adenovirus or retrovirus.

### 2. Abbreviations

The following abbreviations are used throughout the application:
nt=nucleotide
nts=nucleotides
aa=amino acid(s)
kb=kilobase(s) or kilobase pair(s)
kDa=kilodalton(s)
d=day
h=hour
s=seconds

### 3. Nucleic Acid Molecules that Enhance VEGF Expression

The present invention stems at least in part from the discovery that the untranslated regions of VEGF comprise novel control elements each represented by formula (I) as defined above, which reduce the expression of VEGF. Not wishing to be bound by any one particular theory or mode of operation, it is proposed that these control elements reduce the stability of VEGF transcripts by binding or otherwise interacting with a transcript-destabilizing protein. In support of this hypothesis, oligonucleotides comprising control elements of the present invention were found to mediate a 2-fold increase in the amount of VEGF protein and an about 1.25- to 1.5-fold increase in the abundance of VEGF mRNA in VEGF-expressing host cells as compared to a control oligonucleotide that did not contain those elements. Since levels of mRNA are determined by the equilibrium that exists between synthesis and degradation, an increase in stability will reduce degradation and cause an equilibrium shift resulting in higher levels of mRNA being present without an increase in synthesis. The improved mRNA stability, and hence the increased half-life, will result in a proportionally greater amount of protein produced per molecule of mRNA. Thus, in one aspect, the nucleic acid molecules of the present invention are designed to include one or more of these control elements, for example, to compete with endogenous VEGF transcripts for binding to a mRNA destabilizing-protein, or to bind endogenous VEGF transcripts so as to prevent binding of the mRNA destabilizing protein to the transcripts, to thereby increase the amount of VEGF in a host cell or tissue in which VEGF is expressible. Such increase in VEGF expression finds utility in a range of applications including the stimulation of angiogenesis and vascular growth for the treatment of ischemic conditions. These nucleic acid molecules are typically selected from oligonucleotides that comprise at least one control element represented by formula (I) as defined above or polynucleotides from which at least one such control element is expressible.

In some embodiments, the nucleic acid molecules of the present invention comprise at least one nucleotide sequence selected from any one of AGGGG [SEQ ID NO: 1], TGGGG [SEQ ID NO:2] or UGGGG [SEQ ID NO:3]. Generally, the nucleotide sequence will have a length from at least 5 to about 1000 nucleotides. In embodiments where the nucleic acid molecule is an oligonucleotide the oligonucleotide typically comprises at least 5, 6, 7, 8, 9 or 10 to about 15, 20, 25, 30, 35, 40, 50, 60, 70, 80, 90 or 100 nucleotides, and usually comprises at least 10, 11, 12, 13, 14, 15, 16, 17, 18, 19 or 20 nucleotides.

In some embodiments, the oligonucleotide has a length of 12 nucleotides, illustrative examples of which have one of the sequences:5′-GGCTTGGGGCAG-3′ [SEQ ID NO:9]; 5′-CTGGGGGCTAGC-3′ [SEQ ID NO:10]; 5′-GGCTrGGGGAGA-3′ [SEQ ID NO: 11]; and 5′-TGCTTTTGGGGG-3′ [SEQ ID NO:12].

In other embodiments, the oligonucleotide has a length of 13 nucleotides, illustrative examples of which have one of the sequences:5′-GGGCAGGGGCCGG-3′ [SEQ ID NO: 13]; 5′-GGGTGGAGGGGGT-3′ [SEQ ID NO: 14]; 5′-GGAGGGGGAGGAG-3′ [SEQ ED NO: 15]; and 5′-GAGGAGAGGGGGC-3′ [SEQ ID NO: 16].

In still other embodiments, the oligonucleotide has a length of 14 nucleotides, illustrative examples of which have one of the sequences:5′-TGGGAGGGGAATGT-3′ [SEQ ID NO:17]; 5′-GGGCATGGGGGCAA-3′ [SEQ ID NO:18]; 5′-AGGAGTTTGGGGAG-3′ [SEQ ID NO:19]; and 5′-TGGTGGGGCCAGGG-3′ [SEQ ID NO:20].

In still other embodiments, the oligonucleotide has a length of 15 nucleotides, illustrative examples of which have one of the sequences:5′-TGGGGAGCTTCAGGA-3′ [SEQ ID NO:21]; 5′-GCTrrGGGGATTCCC-3′ [SEQ ID NO:22]; 5′-TCGCCCCCAGGGGCA-3′ [SEQ ID NO:23]; and 5′-AATTGTGGGGAAAAG-3′ [SEQ ID NO:24].

In still other embodiments, the oligonucleotide has a length of 16 nucleotides, illustrative examples of which have one of the sequences:5′-CGGAGGCTTGGGGCAG-3′ [SEQ ID NO:25]; 5′-GCTGGGGGCTAGCACC-3′ [SEQ ID NO:26]; 5′-CGACGGCTTGGGGAGA-3′ [SEQ ID NO:27]; and 5′-ACAGGGGCAAAGTGAG-3′ [SEQ ID NO:28].

In still other embodiments, the oligonucleotide has a length of 17 nucleotides, illustrative examples of which have one of the sequences:5′-GCTTTTGGGGGTGACCG-3′ [SEQ ID NO:29]; 5′-AGCCGCGGGCAGQGGCC-3′ [SEQ ID NO:30]; 5′-GGTGGAGjGGGTCGG-3′ [SEQ ID NO:31]; and 5′-AGGAGGGGGAGGAGGAA-3′ [SEQ ID NO:32].

In still other embodiments, the oligonucleotide has a length of 18 nucleotides, illustrative examples of which have one of the sequences:5′-AGAGGGGGCCGCAGTGGC-3′ [SEQ ID NO:33]; 5′-CTTGAGTTGGGAGGGGAA-3′ [SEQ ID NO:34]; 5′-TTGGTGGGGCCAGGGTCC-3′ [SEQ ID NO:35]; and 5′-GCATGGGGGCAAATATGA-3′ [SEQ ID NO:36].

In certain examples, oligonucleotides are designed to comprise at least 2, 3, 4, 5, 6, 7, 8, 9, 10, 11, 12, 13, 14, 15, 16, 17, 18, 19 or 20 control elements represented by the formula (I), which are suitably but not exclusively selected from any one of AGGGG [SEQ ID NO: 1], TGGGG [SEQ ID NO:2] or UGGGG [SEQ ID NO:3].

The invention also contemplates derivatives of the oligonucleotides, for example their salts, in particular their physiologically tolerated salts. Salts and physiologically tolerated salts are described, for example, in Remingtons Pharmaceuticals Science (1985) Mack Publishing Company, Easton, Pa. (page 1418). Derivatives also relate to modified oligonucleotides which have one or more modifications (for example, at particular nucleoside positions and/or at particular internucleoside bridges, oligonucleotide analogues (for example, Polyamide-Nucleic Acids (PNAs), Phosphonic acid monoester nucleic acids (PHONAs=PMENAs), oligonucleotide chimeras (for example, consisting of a DNA- and a PNA-part or consisting of a DNA- and a PHONA-part)). In certain embodiments, the oligonucleotide is modified in order to improve its properties, for example, in order to increase its resistance to nucleases or to make it resistant against nucleases, to improve its binding affinity to an mRNA destabilizing protein, or in order to increase its cellular uptake.

Thus, the present invention advantageously relates to an oligonucleotide which has a particular sequence as outlined above and which has in addition one or more chemical modifications in comparison to a “natural” nucleic acid. For example, DNA, which is composed of the “natural” nucleosides deoxyadenosine (adenine +β-D-2′deoxyribose), deoxyguanosine (guanine +β-D-2′-deoxyribose), deoxycytidine (cytosine +β-D-2′-deoxyribose) and thymidine (thymine +β-D-2′-deoxyribose) is linked via phosphodiester internucleoside bridges. The oligonucleotide can have one or more modifications of the same type and/or modifications of a different type; each type of modification can independently be selected from the types of modifications known to be used for modifying oligonucleotides. For example, in comparison to natural DNA a phosphodiester internucleoside bridge, a β-D-2′-deoxyribose unit and/or a natural nucleoside base (adenine, guanine, cytosine, thymine) can be modified or replaced, respectively. An oligonucleotide according to the invention can have one or more modifications, wherein each modification is located at a particular phosphodiester internucleoside bridge and/or at a particular β-D-2′-deoxyribose unit and/or at a particular natural nucleoside base position in comparison to an oligonucleotide of the same sequence which is composed of natural DNA.

Specific examples of oligonucleotides useful in this invention include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Examples of chemical modifications are known to the skilled person and are described, for example, in E. Uhlmann and A. Peyman, Chemical Reviews 90 (1990) 543 and “Protocols for Oligonucleotides and Analogs” Synthesis and Properties & Synthesis and Analytical Techniques, S. Agrawal, Ed, Humana Press, Totowa, USA 1993 and S. T. Crooke, F. Bennet, Ann. Rev. Pharmacol. Toxicol. 36 (1996) 107-129; J. Hunniker and C. Leuman (1995) Mod. Synt. Methods, 7, 331-417.

Representative disclosures that teach the preparation of phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

Illustrative examples of modified oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative disclosures that teach the preparation of these oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In other embodiments, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a “peptide” or “polyamide” nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative disclosures that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

In certain embodiments, the oligonucleotides comprise one or more modifications and wherein each modification is independently selected from: a) the replacement of a phosphodiester internucleoside bridge located at the 3′- and/or the 5′-end of a nucleoside by a modified internucleoside bridge; b) the replacement of phosphodiester bridge located at the 3′- and/or the 5′-end of a nucleoside by a dephospho bridge; c) the replacement of a sugar phosphate unit from the sugar phosphate backbone by another unit; d) the replacement of a R-D-2′-deoxyribose unit by a modified sugar unit; e) the replacement of a natural nucleoside base by a modified nucleoside base; f) the conjugation to a molecule which influences the properties of the oligonucleotide; g) the conjugation to a 2′5′-linked oligoadenylate or a derivative thereof, optionally via an appropriate linker, and h) the introduction of a 3′-3′ and/or a 5′-5′ inversion at the 3′ and/or the 5′ end of the oligonucleotide.

More detailed examples for the chemical modification of an oligonucleotide are:

a) the replacement of a phosphodiester internucleoside bridge located at the 3′- and/or the 5′-end of a nucleoside by a modified internucleoside bridge, wherein the modified internucleoside bridge is selected for example from phosphorothioate, phosphorodithioate, NR¹R¹′-phosphoramidate, boranophosphate, phosphate-(C₁-C₂₁)—O-alkyl ester, phosphate-[(C₆-C₁₂)aryl-((C₁-C₂₁-)—O-alkyl]ester, (C₁-C₈)alkyl-phosphonate and/or (C₆-C₁₂)-arylphosphonate bridges, (C₇-C₁₂)-.alpha.-hydroxymethyl-aryl (for example, disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R¹′ are, independently of each other, hydrogen, (C₁-C₁₈)-alkyl, (C₆-C₂₀)-aryl, (C₆-C₁₄)-aryl-(C₁-C₈)-alkyl, suitably hydrogen, (C₁-C₈)-alkyl, preferably (C₁-C₄)-alkyl and/or methoxyethyl, or

R¹R¹′ form, together with the nitrogen atom carrying them, a 5-6-membered heterocyclic ring which can additionally contain a further heteroatom from the group O, S and N;

b) the replacement of a phosphodiester bridge located at the 3′- and/or the 5′-end of a nucleoside by a dephospho bridge (dephospho bridges are described, for example, in Uhlmann, E. and Peyman, A. in “Methods in Molecular Biology,” Vol. 20, “Protocols for Oligonucleotides and Analogs,” S. Agrawal, Ed., Humana Press, Totowa 1993, Chapter 16, 355 ff), wherein a dephospho bridge is selected for example from the dephospho bridges formacetal, 3′-thioformacetal, methylhydroxylamine, oxime, methylenedimethyl-hydrazo, dimethylenesulfone and/or silyl groups;

c) the replacement of a sugar phosphate unit (β-D-2′-deoxyribose and phosphodiester internucleoside bridge together form a sugar phosphate unit) from the sugar phosphate backbone (sugar phosphate backbone is composed of sugar phosphate units) by another unit, wherein the other unit is selected from:

(i) a “morpholino-derivative” oligomer (as described, for example, in E. P. Stirchak et al., Nucleic Acids Res. 17 (1989) 6129), that is for example, the replacement by a morpholino-derivative unit;

(ii) a polyamide nucleic acid (“PNA”) (as described for example, in P. E. Nielsen et al., Bioconj. Chem. 5 (1994) 3 and in EP 0672677 A2), that is for example, the replacement by a PNA backbone unit, for example, by 2-aminoethylglycine;

(iii) a phosphonic acid monoester nucleic acid (“PHONA”) (as described for example, in Peyman et al., Angew. Chem. Int. Ed. Engl. 35 (1996) 2632-2638 and in EP 0739898 A2), that is for example, the replacement by a PHONA backbone unit;

d) the replacement of a β-D-2′-deoxyribose unit by a modified sugar unit, wherein the modified sugar unit is selected for example from β-D-ribose, α-D-2′-deoxyribose, L-2′-deoxyribose, 2′-F-2′-deoxyribose, 2′-O—(C₁-C₆)alkyl-ribose, suitably 2′-O—(C₁-C₆)alkyl-ribose is 2′-O-methylribose, 2′-O—(C₁-C₆)alkenyl-ribose, 2′-[O—(C₁-C₆)alkyl-O—(—C₁-C₆)alkyl]-ribose, 2′—NH₂-2′-deoxyribose, β-D-xylo-furanose, α-arabinofuranose, 2,4-dideoxy-β-D-ery-thro-hexo-pyranose, and carbocyclic (described, for example, in Froehler, J. Am. Chem. SQc. 114 (1992) 8320) and/or open-chain sugar analogs (described, for example, in Vandendriessche et al., Tetrahedron 49 (1993) 7223) and/or bicyclosugar analogs (described, for example, in M. Tarkov et al., Helv. Chim. Acta 76 (1993) 481);

e) the replacement of a natural nucleoside base by a modified nucleoside base, wherein the modified nucleoside base is for example selected from uracil, hypoxanthine, 5-(hydroxymethyl)uracil, N²-Dimethylguanosine, pseudouracil, 5-(hydroxymethyl)uracil, 5-aminouracil, dihydrouracil, 5-fluorouracil, 5-fluorocytosine, 5-chlorouracil, 5-chlorocytosine, 5-bromouracil, 5-bromocytosine, 2,4-diaminopurine, 8-azapurine, a substituted 7-deazapurine, preferably 7-deaza-7-substituted and/or 7-deaza-8-substituted purine or other modifications of a natural nucleoside bases, (modified nucleoside bases are for example, described in EP 0 710 667 A2 and EP 0 680 969 A2);

f) the conjugation to a molecule which influences the properties of the oligonucleotide, wherein the conjugation of the oligonucleotide to one or more molecules which (favourably) influence the properties of the oligonucleotide (for example, the ability of the oligonucleotide to penetrate, the cell membrane or to enter a cell, the stability against nucleases, the affinity for mRNA destabilising protein or the pharmacokinetics of the oligonucleotide), wherein examples for molecules that can be conjugated to an oligonucleotide are polylysine, intercalating agents such as pyrene, acridine, phenazine or phenanthridine, fluorescent agents such as fluorescein, crosslinking agents such as psoralen or azidoproflavin, lipophilic molecules such as (C₁₂-C₂₀)-alkyl, lipids such as 1,2-dihexadecyl-rac-glycerol, steroids such as cholesterol or testosterone, vitamins such as vitamin E, poly- or oligoethylene glycol suitably linked to the oligonucleotide via a phosphate group (for example, triethylenglycolphosphate, hexaethylenglycolphosphate), (C₁₂-C₁₈)-alkyl phosphate diesters and/or O—CH₂—CH(OH)—O—(C₁₂-C₁₈)-alkyl, these molecules can be conjugated at the 5′ end and/or the 3′ end and/or within the sequence, for example, to a nucleoside base in order to generate an oligonucleotide conjugate; processes for preparing an oligonucleotide conjugate are known to the skilled person and are described, for example, in Uhlmann, E. & Peyman, A., Chem. Rev. 90 (1990) 543, M. Manoharan in “Antisense Research and Applications,” Crooke and Lebleu, Eds., CRC Press, Boca Raton, 1993, Chapter 17, p. 303 ff. and EP-A 0 552 766;

g) the conjugation to a 2′5′-linked oligoadenylate, suitably via an appropriate linker molecule, wherein the 2′5′-linked oligoadenylate is for example selected from 2′5′-linked triadenylate, 2′5′-linked tetraadenylate, 2′5′-linked pentaadenylate, 2′5′-linked hexaadenyltat or 2′5′-linked heptaadenylat molecules and derivatives thereof, wherein a 2′5′-linked oligoadenylate derivative is for example Cordycepin (2′5′-linked 3′-deoxy adenylate) and wherein an example for an appropriate linker is triethylenglycol and wherein the 5′-end of the 2═5′-linked oligoadenylate must bear a phosphate, diphosphate or triphosphate residue in which one or several oxygen atoms can be replaced for example, by sulfur atoms, wherein the substitution by a phosphate or thiophosphate residue is desirable; and

h) the introduction of a 3′-3′ and/or a 5′-5′ inversion at the 3′ and/or the 5′ end of the oligonucleotide, wherein this type of chemical modification is known to the skilled person and is described, for example, in M. Koga et al, J. Org. Chem. 56 (1991) 3757, EP 0 464 638 and EP 0 593 901.

The replacement of a sugar phosphate unit from the sugar phosphate backbone by another unit, which is for example, a PNA backbone unit or a PHONA backbone unit, is suitably the replacement of a nucleotide by, for example, a PNA unit or a PHONA unit, which already comprises natural nucleoside bases and/or modified nucleoside bases, for example, one of the modified nucleoside bases from uracil, hypoxanthine, 5-(hydroxymethyl)uracil, N²-Dimethylguanosine, pseudouracil, S-(hydroxy-methyl)uracil, 5-aminouracil, pseudouracil, dihydrouracil, 5-fluorouracil, 5-fluorocytosine, 5-chlorouracil, 5-chlorocytosine, 5-bromouracil, 5-bromocytosine, 2,4-diamino-purine, 8-azapurine, a substituted 7-deazapurine, preferably 7-deaza-7-substituted and/or 7-deaza-8-substituted purine or other modifications of a natural nucleoside bases, (modified nucleoside bases are described, for example, in EP 0 710 667 A2 and EP 0 680 969 A2).

In certain examples, one or more phosphodiester internucleoside bridges within the oligonucleotide sequence are modified, desirably one or more phosphodiester internucleoside bridges are replaced by phosphorothioate internucleoside bridges and/or (C₆-C₁₂)aryl phosphonate internucleoside bridges, suitably by α-hydroxybenzyl phosphonate bridges in which the benzyl group is preferably substituted, for example, with nitro, methyl, halogen.

In other examples, one or more sugar phosphate units from the sugar-phosphate backbone are replaced by PNA backbone units, suitably by 2-aminoethylglycine units. Desirably, the sugar phosphate units which are replaced are connected together at least to a certain extent If desired, not all sugar phosphate units are uniformly replaced in the olignucleotide. In illustrative examples of this type, the oligonucleotides chimeric and are composed of one or more PNA parts and one or more DNA parts. For such chimeric oligonucleotides, for example the following non-limiting examples of modification patterns are possible: DNA-PNA, PNA-DNA, DNA-PNA-DNA, PNA-DNA-PNA, DNA-PNA-DNA-PNA, PNA-DNA-PNA-DNA. Comparable patterns would be possible for chimeric molecules composed of DNA parts and PHONA parts, for example, DNA-PHONA, PHONA-DNA, DNA-PHONA-DNA, PHONA-DNA-PHONA, DNA-PHONA-DNA-PHONA, PHONA-DNA-PHONA-DNA. In addition of course, chimeric molecules comprising three different parts like DNA part(s), PHONA part(s) and PNA part(s) are possible. Preferably the invention relates to an oligonucleotide which comprises in addition at least one other type of modification.

The principle of partially modified oligonucleotides is described for example, in A. Peyman, E. Uhlmann, Biol. Chem. Hoppe-Seyler, 377 (1996) 67-70 and in EP 0 653 439. In this case, 1-5 terminal nucleotide units at the 5′ end/or and at the 3′ end are protected, for example, the phosphodiester internucleoside bridges located at the 3′ and/or the 5′ end of the corresponding nucleosides are for example replaced by phosphorothioate internucleoside bridges. In addition, at least one internal pyrimidine nucleoside (or nucleotide respectively) position is typically modified; desirably the 3′ and/or the 5′ internucleoside bridge(s) of a pyrimidine nucleoside is/are modified/replaced, for example by phosphorothioate internucleoside bridge(s). Partially modified oligonucleotides exhibit particularly advantageous properties; for example they exhibit a particularly high degree of nuclease stability in association with minimal modification. Partially modified oligonucleotides also show a higher binding affinity than all-phosphorothioates.

As an alternative to the oligonucleotides described above, the present invention also contemplates the use of nucleic acid constructs that comprise a polynucleotide that is transcribed by the cell machinery to give rise to a transcript that comprises at least one nucleotide sequence represented by formula (I) as defined above. In some embodiments, the nucleotide sequence comprises a nucleic acid sequence corresponding to an untranslated region (UTR) of a VEGF transcript or portion thereof that comprises at least one nucleotide sequence represented by formula (I) as defined above. Portions of a VEGF UTR may range from at least about at least 12, 13, 14, 15, 16, 17, 18, 19, 20, 25, 30 or 35 to about 100, 150, 200, 300, 400 or 500 contiguous nucleotides, or almost up to the full-length 5′-UTR and 3 U of a VEGF gene as set forth, for example in SEQ ID NO: 6 and 7, respectively.

The present invention also contemplates nucleic acid molecules that comprise sequence regions that are about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 76%, 75%, 74%, 73%, 72%, 71% or even 70% identical to a portion of one of the VEGF UTRs, so long as the resulting degenerate nucleotide sequence retains sufficient homology so that it destabilizes a transcript to which it is operably connected to thereby increase the amount of VEGF in a host cell that expresses VEGF. Typically, the degenerate nucleotide sequence will comprise at least one control element represented by formula (I) as herein defined. Accordingly, the present invention also encompasses the complement of a nucleotide sequence that hybridize to at least a portion of the VEGF 5′-UTR or 3′-UTR under at least low stringency conditions, preferably under at least medium stringency conditions and more preferably under high stringency conditions.

As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 10/Q Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, an isolated nucleic acid molecule of the invention hybridizes under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8). In general, the Tₘ of a perfectly matched duplex of DNA may be predicted as an approximation by the formula:

Tₘ=81.5+16.6(log₁₀M)+0.41(% G+C)−0.63 (% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ₋₁₅° C. for high stringency, or Tₘ−30° C. for medium stringency.

In one example of a hybridization procedure, a membrane (e.g., a nitrocellulose membrane or a nylon membrane) containing immobilized DNA is hybridized overnight at 42° C. in a hybridization buffer (50% deionised formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

A nucleic acid molecule according to the invention increases VEGF protein expression by about 55%, 65%, 75%, 85%, 90%, 95%, 100% or more relative to control cells or control tissue, e.g. the amount of secreted VEGF is increased by about 55%, 65%, 75%, 85%, 90%, 95%, 100% or more when the cell is treated with a nucleic acid molecule according to the invention at a concentration generally from about 0.1, 0.2, 0.3, 0.4 or 0.5 μM to about 10, 20, 30, 40 or 50 μM, and usually at a concentration of about 1 μM or less. Suitably, a nucleic acid molecule of the invention can efficiently increase the expression of VEGF (isoforms) in an animal cell and/or has the ability to stimulate angiogenesis or vascular growth in vertebrates.

In certain embodiments, nucleic acid molecules of the invention are in the form of nucleic acid constructs (e.g., expression vectors such as but not limited to viral vectors, such as retro-, adeno- or adeno-associated or lentiviral vectors). In some embodiments, such constructs possess a promoter that is operably connected to a polynucleotide that encodes a transcript comprising at least one nucleotide sequence represented by formula (I) as defined above: The promoter may be inducible or constitutive, and, optionally, tissue-specific. The promoter may be, for example, viral or mammalian in origin. In some embodiments, a nucleic acid construct is used in which the promoter-polynucleotide cassette (and any other desired sequences) is flanked by regions that promote homologous recombination at a desired site within the genome of a subject, thus providing for intra-chromosomal expression of the polynucleotide. See e.g., Koller and Smithies, 1989. Proc Natl Acad Sci USA 86: 8932-8935. In other embodiments, the nucleic acid construct that is delivered remains episomal and induces an endogenous and otherwise silent gene.

In one illustrative embodiment, retroviruses provide a convenient and effective platform for gene delivery systems. A nucleotide sequence for which at least one control element of the present invention is expressible can be inserted into a vector and packaged in retroviral particles using techniques known in the art. The recombinant virus can then be isolated and delivered to a subject. Several illustrative retroviral systems have been described examples of which include: U.S. Pat. No. 5,219,740; Miller and Rosman, 1989, Bio Techniques 7: 980-990; Miller, A. D., 1990, Human Gene Therapy 1: 5-14; Scarpa et al., 1991, Virology 180: 849-852; Burns et al., 1993, Proc. Natl. Acad. Sci. USA 90: 8033-8037; and Boris-Lawrie and Temin, 1993, Cur. Opin. Genet. Develop. 3: 102-109).

In addition, several illustrative adenovirus-based systems have also been described. Unlike retroviruses which integrate into the host genome, adenoviruses persist extrachromosomally thus minimising the risks associated with insertional mutagenesis (see, e.g., Haj-Ahmad and Graham, 1986, J. Virol. 57: 267-274; Bett et al., 1993, J. Virol. 67: 5911-5921; Mittereder et al., 1994, Human Gene Therapy 5: 717-729; Seth et al., 1994, J. Virol. 68: 933-940,; Barr et al., 1994, Gene Therapy 1: 51-58; Berkner, K. L., 1988, Bio Techniques 6: 616-629; and Rich et al., 1993, Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., 1988, Molec. Cell. Biol. 8: 3988-3996; Vincent et al., 1990, Vaccines 90, Cold Spring Harbor Laboratory Press; Carter, B. J., 1992, Current Opinion in Biotechnology 3: 533-539; Muzyczka, N., 1992, Current Topics in Microbiol. and Immunol 158: 97-129; Kotin, R. M., 1994, Human Gene Therapy 5: 793-801; Shelling and Smith, 1994, Gene Therapy 1: 165-169; and Zhou et al., 1994, J. Exp. Med, 179: 1867-1875.

Alternatively, avipoxviruses, such as the fowlpox and canarypox viruses, can also be used to deliver the coding sequences of interest. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al., J. Biol. Chem. 268:6866-69, 1993; and Wagner et al., Proc. Natl. Acad. Sci. USA 89:6099-6103, 1992, can also be used for gene delivery under the invention.

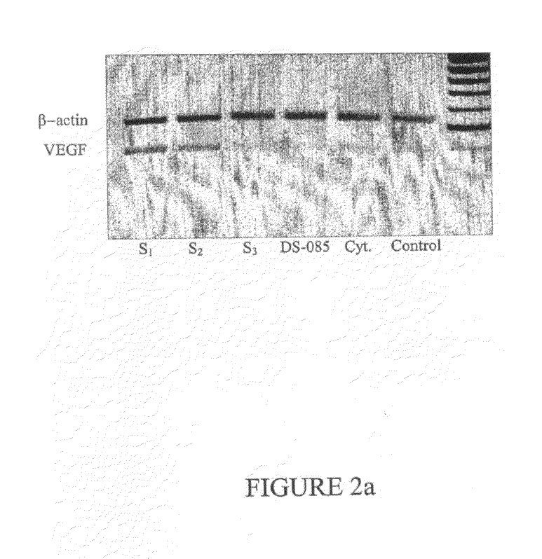

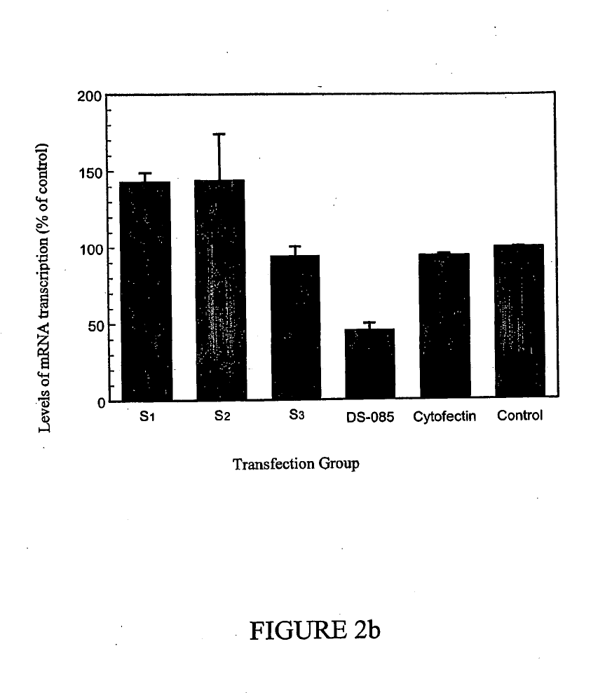

In other illustrative embodiments, lentiviral vectors are employed to deliver the control element-expressing polynucleotide into selected cells or tissues. Typically, these vectors comprise a 5′ lentiviral LTR, a tRNA binding site, a packaging signal, a promoter operably linked to one or more genes of interest, an origin of second strand DNA synthesis and a 3′ entiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilized within the context of the present invention, including for example, lentiviruses selected from the group consisting of HIV, HIV-1, HIV-2, FIV, BIV, EIAV, MVV, CAEV, and SIV. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurons, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-1-Derived Lentiviral Vectors. In: Trono, D. (Ed.), Lentiviral Vectors, Springer-Verlag, Berlin, Heidelberg, New York, 2002, pp. 31-52). An example of the current generation vectors is outlined in FIG. 2 of a review by Lois et al. (Lois, C., Hong, E. J., Pease, S., Brown, E. J., and Baltimore, D., Germline transmission and tissue-specific expression of transgenes delivered by lentiviral vectors, Science, 295 (2002) 868-872).

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

### 4. Modes of Delivery

Delivery of the nucleic acid molecules into a patient may be either direct (i.e., the patient is directly exposed to the nucleic acid or nucleic acid-containing vector) or indirect (i.e., cells are first contacted with the nucleic acid in vitro, then transplanted into the patient). These two approaches are known, respectively, as in vivo or er vivo gene therapy. In a specific embodiment of the present invention, a nucleic acid is directly administered in vivo, where it is expressed to produce the encoded product. This may be accomplished by any of numerous methods known in the art including, but not limited to, constructing the nucleic acid as part of an appropriate nucleic acid expression vector, as discussed above and administering the same in a manner such that it becomes intracellular (e g, by infection using a defective or attenuated retroviral or other viral vector; see U.S. Pat. No. 4,980,286); directly injecting naked DNA; using microparticle bombardment (e.g., a “Gene Gun®”; Biolistic, DuPont); coating the nucleic acids with lipids; using associated cell-surface receptors/transfecting agents; encapsulating in liposomes, microparticles, or microcapsules; administering it in dendrimer form or in linkage to a peptide that is known to enter the nucleus; or by administering it in linkage to a ligand predisposed to receptor-mediated endocytosis (see, e.g., Wu and Wu, 1987. J Biol Chem 262: 4429-4432), which can be used to “target” cell types that specifically express the receptors of interest, etc.

An additional approach to gene therapy in the practice of the present invention involves transferring a gene into cells in in vitro tissue culture by such methods as electroporation, lipofection, calcium phosphate-mediated transfection, viral infection, or the like. Generally, the methodology of transfer includes the concomitant transfer of a selectable marker to the cells. The cells are then placed under selection pressure (e.g, antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, but not limited to: transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. The chosen technique should provide for the stable transfer of the nucleic acid to the cell, such that the nucleic acid is expressible by the cell. Desirably, the transferred nucleic acid is heritable and expressible by the cell progeny. In other embodiments, the transferred nucleic acid remains episomal and induces the expression of the otherwise silent endogenous nucleic acid. In some embodiments, the resulting recombinant cells may be delivered to a patient by various methods known within the art including, but not limited to, injection of epithelial cells (e g, subcutaneously), application of recombinant skin cells as a skin graft onto the patient, and intravenous injection of recombinant blood cells (e.g., hematopoietic stem or progenitor cells) or liver cells. The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g., as obtained from bone marrow, umbilical cord blood, peripheral blood, fetal liver, and the like. In a preferred embodiment, the cells utilized for gene therapy are autologous to the patient. In certain embodiments, the invention contemplates the use of liposomes, nanocapsules, microparticles, microspheres, lipid particles, vesicles, and the like, for the introduction of the nucleic acid molecules of the present invention into suitable host cells. In particular, the nucleic acid molecules of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acid molecules disclosed herein. The formation and use of liposomes is generally known to those of skill in the art. Liposomes have been developed with improved serum stability and circulation half-lives (see U.S. Pat. No. 5,741,516). Further, various methods of liposome and liposome like preparations as potential drug carriers have been reviewed (U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are typically formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The preparation and use of liposomes, e.g., using the certain the commercially available transfection reagents DOTAP (Roche Diagnostics), Lipofectin, Lipofectam, and Transfectam, is well known in the art. Other methods of obtaining liposomes include the use of Sendai virus or of other viruses. Examples of publications disclosing oligonucleotide transfer into cells using the liposome technique are e.g., Meyer et al. [J. Biol. Chem. 273, 15621-7 (1998)], Kita and Saito [Int. J. Cancer 80, 553-8 (1999)], Nakamura et al. [Gene Ther. 5, 1455-61 (1998)] Abe et al. [Antivir. Chem. Chemother. 9, 253-62 (1998)], Soni et al. [Hepatology, 28, 1402-10 (1998)], Bai et al. [Ann. Thorac. Surg. 66, 814-9 (1998) and see also discussion in the same journal p. 819-20], Bochot et al. [Pharm. Res. 15, 1364-9 (1998)], Noguchi et al. [FEBS Lett. 433, 169-73 (1998)], Yang et al. [Circ. Res. 83, 552-9 (1998)], Kanamaru et al. [J. Drug Target. 5, 235-46 (1998)] and references therein. The use of Lipofectin in liposome-mediated oligonucleotide uptake is described in Sugawa et al. [J. Neurooncol. 39, 237-44 (1998)]. The use of fusogenic cationic-lipid-reconstituted influenza virus envelopes (cationic virosomes) is described in Waelti et al. [Int. J. Cancer, 77, 728-33 (1998)].

The above-mentioned cationic or nonionic lipid agents not only serve to enhance uptake of oligonucleotides into cells, but also improve the stability of oligonucleotides that have been taken up by the cell.

Alternatively, the invention provides for pharmaceutically acceptable nanocapsule formulations of the nucleic cid molecules of the present invention. Nanocapsules can generally entrap compounds in a stable and reproducible way. To avoid side effects due to intracellular polymeric overloading, such ultrafine particles (sized around 0.1 μm) are typically designed using polymers able to be degraded in vivo. Biodegradable polyalkyl-cyanoacrylate nanoparticles that meet these requirements are contemplated for use in the present invention, and such particles are easily made, as described for example in U.S. Pat. No. 5,145,684. In particular, methods of oligonucleotide delivery to a target cell using either nanoparticles or nanospheres (Schwab et al., 1994; Proc. Natl. Acad. Sci. USA, 91(22):10460-10464; Truong-Le et al., 1998, Hum. Gene Ther., 9(12):1709-1717) are also particularly contemplated to be useful in formulating the disclosed compositions for administration to an animal, and to a human in particular.

In other embodiments, the invention provides for dendrimer formulations of the nucleic cid molecules of the present invention, which typically comprise two or more nucleic acid molecules linked to a central branching molecule. Methods of making “oligonucleotide dendrimers” are generally known in the art. (See, e.g., U.S. Pat. No. 6,455,071; U.S. Pat. No. 6,274,723; Azhayeva et al., Nucleic Acids Res. 23:1170-1176, 1995; Horn and Urdea, Nucleic Acids Res. 17:6959-6967, 1989.) The “branching molecule” can be monomeric or polymeric, and linkage to the branching molecule can be via covalent or non-covalent interactions. Thus, dendrimers according to the present invention can include, for example, a plurality of oligonucleotides linked covalently to a branching molecule such as, e.g., a nucleoside derivative (as described in, e.g., Azhayeva et al., supra; Horn and Urdea, supra) or a phosphoramidite synthon (see, e.g., Shchepinov et al., Nucleic Acids Res. 25:4447-4454, 1997). In other embodiments, the oligonucleotides are linked non-covalently to the branching molecule such as, for example, a nucleic acid polymer by, e.g., hybridization of substantially complementary regions. For example, the branching molecule can be a dimer of two partially single-stranded nucleic acids, linked at an internal region by complementary base pairing and having four single-stranded regions available for linkage to a nucleic acid molecule of the invention (see, e.g., U.S. Pat. No. 6,274,723).

It will be understood, however, that the present invention is not limited to or dependent on any particular mode of administration but instead encompasses all modes of delivery of nucleic acid compositions.

### 5. Pharmaceutical Compositions

In order to be effective, the nucleic acid molecules of the invention, also when comprised in a pharmaceutical composition of the invention, must travel across cell membranes. In general, oligonucleotides have the ability to cross cell membranes, apparently by a saturable uptake mechanism linked to specific receptors. As oligonucleotides are single-stranded molecules, they are to a degree hydrophobic, which enhances passive diffusion through membranes. Modifications may be introduced to an oligonucleotide to improve its ability to cross membranes. For instance, the oligonucleotide molecule may be linked to a group comprising optionally partially unsaturated aliphatic hydrocarbon chain and one or more polar or charged groups such as carboxylic acid groups, ester groups, and alcohol groups. Alternatively, oligonucleotides may be linked to peptide structures, which are suitably membranotropic peptides. Such modified oligonucleotides penetrate membranes more easily, which is critical for their function and may therefore significantly enhance their activity. Palmityl-linked oligonucleotides have been described by Gerster et al. [Anal. Biochem. 262, 177-84 (1998)]. Geraniol-linked oligonucleotides have been described by Shoji et al. [J. Drug Target 5, 261-73 (1998)]. Oligonucleotides linked to peptides, e.g., membranotropic peptides, and their preparation have been described by Soukchareun et al. [Bioconjug. Chem. 9, 466-75 (1998)]. Modifications of antisense molecules or other drugs that target the molecule to certain cells and enhance uptake of the oligonucleotide by said cells are described by Wang, J. [Controlled Release 53, 3948 (1998)].

It will also be understood that, if desired, the nucleic acid compositions disclosed herein may be administered in combination with other agents as well, such as, e.g., proteins or polypeptides or various pharmaceutically-active agents. As long as the composition comprises at least one VEGF expression-enhancing nucleic acid molecule, there is virtually no limit to other components that may also be included, given that the additional agents do not cause a significant adverse effect upon contact with the target cells or host tissues. The nucleic acid molecules may thus be delivered along with various other agents as required in the particular instance.

The formulation of pharmaceutically-acceptable excipients and carrier solutions are well-known to those of skill in the art, as is the development of suitable dosing and treatment regimens for using the particular compositions described herein in a variety of treatment regimens, including, for example, oral, parenteral, intravenous, intranasal, and intramuscular administration and formulation.

Alternatively, the pharmaceutical compositions disclosed herein may be administered parenterally, intravenously, intramuscularly, or even intraperitoneally or directly, for example by instillation, into the target organ as described, for example, in U.S. Pat. No. 5,543,158, U.S. Pat. No. 5,641,515 and U.S. Pat. No. 5,399,363. Solutions of the active compounds as free-base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (see, for example, U.S. Pat. No. 5,466,468). In all cases the form must be sterile and must be fluid to the extent that easy syringability exists. It is desirably stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage may be dissolved in 1 ml of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the therapeutic goods regulatory authority.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

The compositions of the present invention suitable for oral administration may be presented as discrete units such as capsules, sachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste. A tablet may be made by compression or molding, optionally with one or more accessory ingredients. Compressed tablets may be prepared by compressing in a suitable machine the active ingredient in a free-flowing form such as a powder or granules, optionally mixed with a binder (for example, povidone, gelatin, hydroxypropylmethyl cellulose), lubricant, inert diluent, preservative, disintegrant (for example, sodium starch glycolate, cross-linked povidone, cross-linked sodium carboxymethyl cellulose) surface-active or dispersing agent. Moulded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The pharmaceutical compositions of the invention generally comprise a buffering agent, an agent which adjusts the osmolarity thereof, and optionally, one or more carriers, excipients and/or additives as known in the art, e.g., for the purposes of adding flavors, colors, lubrication, or the like to the pharmaceutical composition. A preferred buffering agent is phosphate-buffered saline solution (PBS), which solution is also adjusted for osmolarity.

Carriers may include starch and derivatives thereof, cellulose and derivatives thereof, e.g., microcrystalline cellulose, xantham gum, and the like. Lubricants may include hydrogenated castor oil and the like.

In some embodiments, the pharmaceutical formulation is one lacking a carrier. Such formulations are preferably used for administration by injection, including intravenous injection and instillation.

The invention also relates to a method for the treatment or prevention of an ischemic condition or the reduction, prevention or treatment of ischemia-related tissue damage, comprising administering the nucleic acid molecules of the or a pharmaceutical composition of the invention to a patient in need thereof.

In order that the invention may be readily understood and put into practical effect, particular preferred embodiments will now be described by way of the following non-limiting examples.

### EXAMPLES

### Example 1

### Regulation of VEGF Expression

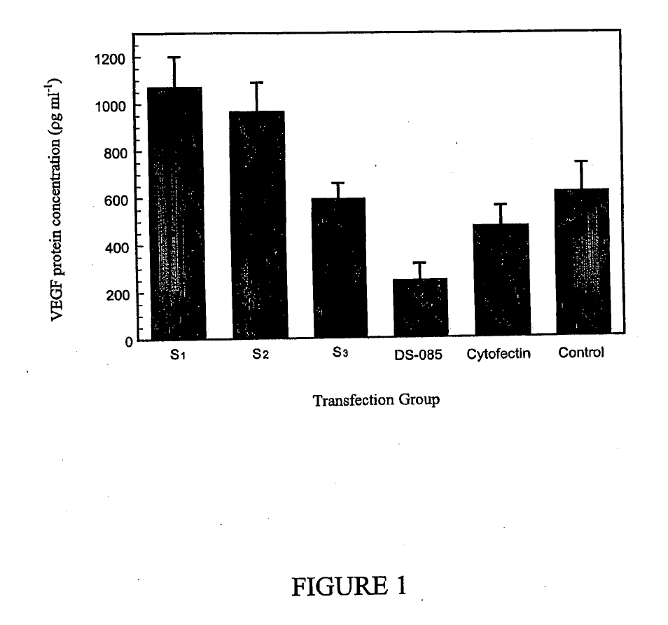

Oligonucleotides S1, S2 S3 and DS-085 (see Materials and Methods) were transfected into the RPE 51 cell line and the effects of VEGF translation and transcription were measured using ELISA and RT-PCR respectively. ELISA (FIG. 1) revealed that both S1 (1073 pg mL⁻¹) and S2 (969 pg ml⁻¹) facilitated a significant (p<<0.01) upregulation of VEGF protein by approximately 2 fold compared to the non-transfected control (578 pg mL⁻¹), oligonucleotide S3 (593 pg mL⁻¹) had no significant effect (P>0.05) transfection agent cytofectin mediated a slight decrease in VEGF expression (508 pg mL⁻¹). However, this result was not found to be significant (p>0.05). Regulation of vascular endothelial growth factor.

To examine the effects on VEGF at the transcriptional level, total RNA was extracted from cells transfected with oligonucleotide S1, S2, S3 and DS-085 and subsequently used as a template for RT-PCR (FIG. 2a). The profile for mRNA levels in the transfected cells as determined by densitometry of the PCR products (FIG. 2b) reflected the protein concentration profile. Transfection with S1 and S2 mediated an increase in the levels of mRNA by a factor of 1.5 compared to the non-transfected control, 24 hours after transfection. However, the increase in mRNA mediated by s1 and S2 was not found to be proportional to the increase in protein concentration. The previously described oligonucleotide DS-085 decreased the level of mRNA by 57.5%, which is directly proportional to the decrease in protein. This indicates that the mechanism of down-regulation by DS-085 is separate to and distinct from the mechanism of up-regulation of protein by s1 and S2. Transfection with the S3 oligonucleotide resulted in no significant effect compared to the control samples, which is the same for the protein concentration. Similarly, transfection with vehicle (Cytofectin™) alone produced a slight decrease (5%) in VEGF mRNA equivalent to that found for the protein reduction and may be reflective of the slight cytotoxic effect known to be associated with Cytofectin™ (22).

### Material and Methods

### Oligonucleotide Design

The 5′-UTR sequence of human VEGF was examined for the presence of homopurine regions that may represent potential regulatory sites. Sense oligonucleotides 1 and 2 (S1 and S2) were subsequently designed to recognize the first and final 16 by respectively of a homopurine homopyrimidine sequence identified from base pair −265 to −223 from the ATG start codon. Sense oligonucleotide (S3) represented the 16 by immediately 5′ to Sense oligonucleotide 1 and was used as a control. Oligonucleotide DS-085 has been previously described (Garrett, 2000). Oligonucleotides were obtained from Proligo (Boulder, Colo., USA) and synthesized with a phosphorothioate (S) backbone.

### In Vitro Oligonucleotide VEGF Inhibition Assay

A Human retinal pigment epithelial (RPE) cell line (RPE 51) was grown in culture and used to assess the effect of various oligonucleotide sequences on the production of VEGF protein and mRNA. Cells were seeded into 2×6 well plates (35 mm diameter) at approximately 4×10⁵ cells per well and allowed to grow in Dulbecco's modified Eagle's medium supplemented with 10% fetal calf serum, 0.5% streptomycin and penicillin at −37° C. and 5% CO₂ until 80% confluent. Cytofectin™ (Gene Therapy Systems, San Diego, Calif., USA) was used as per the manufactures instructions to deliver the oligonucleotides into the cells at a final concentration of 1 μM. Control groups consisted of cells transfected with cytofectin minus an oligonucleotide and null treated cells which were not manipulated in any way. Following transfection, one of the plates was transferred to a CO₂ incubator and grown under normoxic condition at 5% CO₂. The other plate was placed in a hypoxic incubator (2% O2, 5% CO2) and each was grown for 24 hours. Regulation of vascular endothelial growth factor.

After this time the media was extracted from both the normoxic and hypoxic grown cells for enzyme-linked immunosorbent assay (ELISA) using a commercially available kit (CYTELISA™, Cytimmune Sciences, Maryland, USA) to determine the level of VEGF expression. The ELISA was performed as per the manufactures instructions using 100 μL of undiluted culture media. The cells from each well were harvested by trypsinisation and pelleted by centrifugation at 2000 g for 5 minutes. The cell pellet was washed-twice in isotonic saline and resuspended in 250 μL of the same. An OD600 reading was taken to determine cell density, which was used to normalize the VEGF concentration.

Levels of mRNA transcription were determined using RT-PCR. Cells were treated with 600 μL of Trizol™ (QIAGEN, Clifton Hill, Vic, Australia) directly in the culture wells 24 hours post transfection. The lysed suspension was removed to a microfuge tube where chloroform (200 μL) was added and the solution vortexed to ensure complete mixing. The aqueous phase containing the total RNA was removed to a new microfuge tube and one volume of isopropanol added to precipitate the RNA. The RNA was pelleted by centrifugation at 20000 g for 10 minutes and the RNA pellet washed in 70% ethanol. The ethanol was aspirated and the pellet air dried, resuspended in 200 μL of nuclease free water and the concentration determined spectrometrically. An Omniscript™ RT kit (QIAGEN) was used for the production of the first strand cDNA as per the manufactures instructions starting with 200 μg of total RNA using an oligo dT primer in a final volume of 20 μL. Directly from this reaction, 1 μL was used as a template for PCR of an internal VEGF fragment in addition to a β-actinfragment, which was used as an internal control. VEGF-primers consisted of the sense 5′-CATCACGAAGTGGTGAAGTT-3′ and the antisense 5′-AACGCTCCAGGACTTATACC3′. Primers used to amplify β-actin consisted of the sense 5′-AGGCACCAGGGCGTGAT-3′, and the antisense 5′-TTAATGTCACGCACGATITC-3′. Both sets of primers (Proligo) were included with the following reaction components in a final volume of 25 μL; 2.5 μL of 10× reaction buffer, 2 mM MgCl₂, 200 μM of each dNTP, 6 pmoles of each primer and 1 unit of Tth⁺ polymerase (Fisher Biotech, Perth, Wash., Australia). A touch down cycling reaction was used and consisted of an initial denaturing step of 94° C. for 2 minutes followed by seven cycles of 94° C.-10 seconds; 65° C.-10 seconds with a drop of 1° C. per cycle; 72° C.-30 seconds. This was then followed by 41 cycles of 94° C.-10 seconds; 58° C.-10 seconds; 71° C.30 seconds.

### Sample Statistics

The transfection was performed in quadruplet sets for statistical analysis. Results were analysed by one-way ANOVA followed by a post hoc Turkey/Kramer analyses with 99% confidence limits using the GB-Stat™ statistical software package (Dynamic Microsystems, Silver Springs, Md., USA).

### Example 2

### In Vivo Analysis

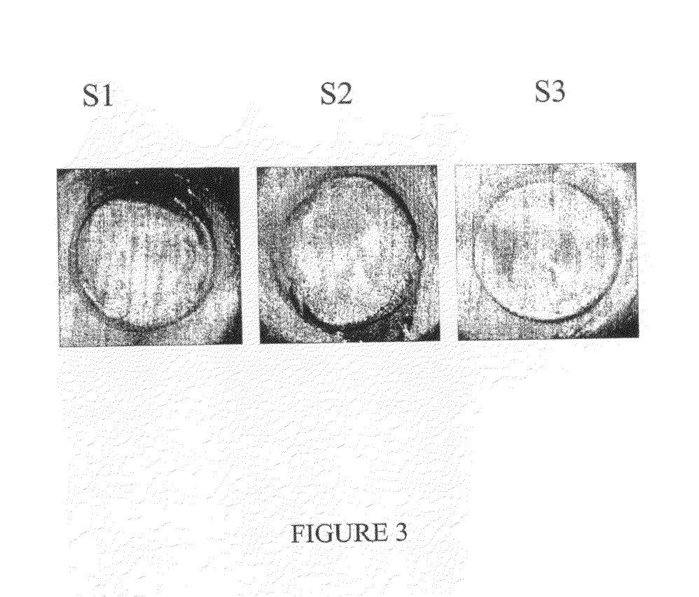

To determine if the in vitro observations would translate into an in vivo effect, the oligonucleotides were injected into the anterior chamber of rat eyes. Subsequent opthalmologic examination showed strong neovascularization in the iris of rat eyes 7 days following injection with S1 and S2, (FIG. 3) but no effect was observed in rat eyes injected with S3 or vehicle. This indicates that the oligonucleotides were able to mediate the up-regulation of VEGF the eye and produce an angiogenic response.

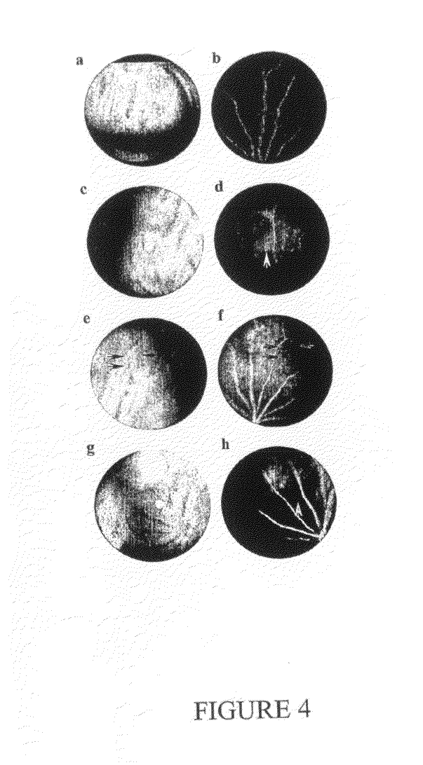

Similarly, when the rats were injected in the sub-retinal space with S1 and S2 a strong angiogenic response was observed when viewed using color fundus photography. Neovascularization occurred some distance from the injection site and appeared as a distinct band extending across the retina (FIG. 4a). Further examination using fluorescein angiography confirmed the formation of new vessels which appears as hyperfluorescence (FIG. 4b) due to the “leaky” nature blood vessels during angiogenesis. In addition, fluorescein angiography revealed the presence of micro aneurisms (FIG. 4c) in eyes injected with S1 and S2. Later examination performed 14 days post injection revealed the occurrence of intra-retinal hemorrhage in eyes injected with s1 and S2 which appears as black spots using color fundus photography (FIG. 4d) and as hypo-fluorescence using fluorescein angiography (FIG. 4e). These results were closely paralleled in the mouse model following subretinal injection where leakiness, micro aneurysms and intraretinal hemorrhage was observed in eyes injected with s1 and S2 7 days post injection. Eyes injected with S3 retained a normal appearance.

### Material and Methods

### Injections and In Vivo Analysis

All animal experiments were performed in accordance with the Animal Use guidelines of the Association for Research in Vision and Opthalmology and were approved by the Animal Ethics Committee of The University of Western Australia. Oligonucleotide delivery to the anterior chamber was carried out on 6-8 week old non-pigmented RCS-rdy+rats which had been anaesthetized by an intramuscular injection of ketamine (50 mg kg⁻¹ body weight) and xylazine (8 mg kg⁻¹ body weight), followed by topical application of proparacaine hydrochloride to the eye. Two and a half μL of a 1 mM oligonucleotide solution or vehicle (PBS containing 10% glycerol) were injected into the anterior chamber of both eyes of each rat via the temporal limbus, using a 32-gauge needle attached to a 5 μL Hamilton syringe, after the same amount of aqueous humor was drained. Opthalmologic examinations of the eyes were performed 7 days post injection and photographed using a slit lamp camera.

Sub retinal injections were performed on 8- to 9-week old nonpigmnented RCS/rdy⁺ rats and C57 Black C6J mice of the same age. The injection technique used has been described previously (21). Briefly, the conjunctiva was cut close to the limbus to expose the sclera, which was then punctured with a 30-gauge needle. A 32-gauge needle was passed through this hole in a tangential direction under an operating microscope. Two uL of oligonucleotide were delivered into the subretinal space of each eye. The needle was kept in the subretinal space for 1 minute, withdrawn gently, and antibiotic ointment applied to the wound site.

### Example 3

### Sequence Comparison

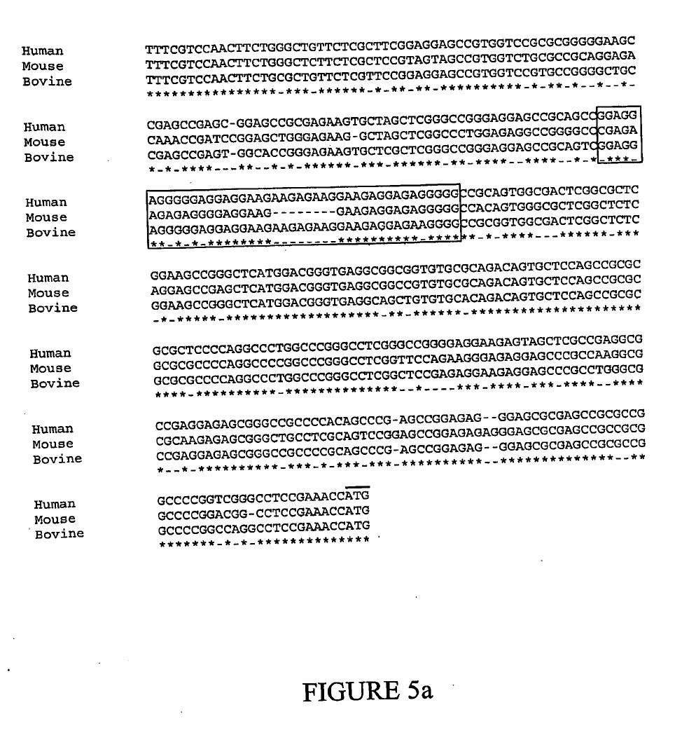

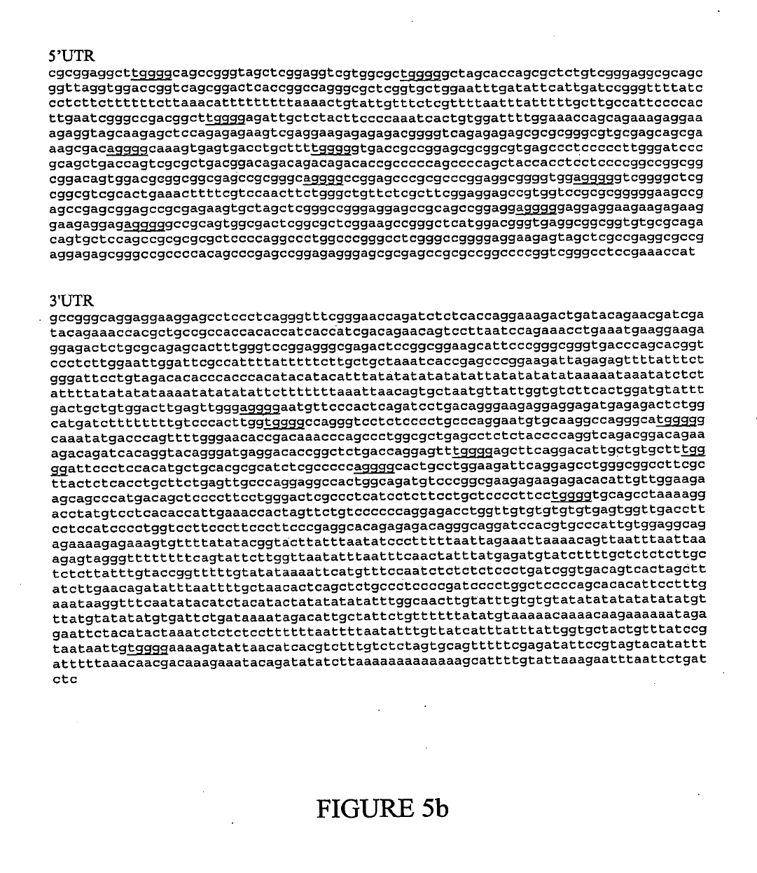

Cross species comparisons of VEGF 5′-UTR sequences between bovine, murine and human has revealed a high level of conservation for the S1 to S2 region between human and bovine but the murine 5′-UTR revealed a complete lack of the S1 sequence (FIG. 5a). No sequence information is available for the 5′-UTR of the rat therefore no direct comparison can be made. Further examination of both the 5′- and 3′-UTR of the human VEGF gene has revealed several possible sites for destabilizing elements (FIG. 5b).

### Discussion of the Examples

Controlled regulation of VEGF in vivo is important in maintaining the health of many tissues and cells types. However, increased levels of VEGF associated with ischemic conditions leads to a variety of angiogenic ocular diseases including diabetic retinopathy and retinopathy of prematurity (23,24), in addition to promoting vasculogenesis in cancerous tissues (25,26). Central to the regulation of VEGF is the presence of both a 5′-UTR, and 3′-UTR, both of which contain many regulatory elements including hypoxia and glucose response elements (27) in addition to stabilizing and destabilizing elements (9). In this study we report on the discovery of a novel control element within the 5′-UTR of the human VEGF gene that may act as a target site for a destabilizing protein in addition to providing further insight into its regulation.

Two sense oligonucleotides (S1 and S2) were designed to resemble a potential regulatory region within the 5′-UTR of the VEGF gene. A third oligonucleotide (S3) was designed as a control and mapped to the 16 by immediately 5′ to S1. Results from the in vitro studies demonstrated that S1 and S2 mediated a 2-fold increase in protein production and up to a 1.5 fold increase in the mRNA translation. This indicates that the sequences in the 5′-UTR represented by S1 and S2 contain regulatory elements involved in the modulation of VEGF production. Possible mechanisms for VEGF protein up-regulation by S1 and S2 include competitive inhibition of either a mRNA destabilizing protein or a transcriptional repressor protein. In the case of the latter, transcriptional repressor proteins have been previously described (19,20) and share a common theme of recognizing variations of a homopurine, GA type sequence consensus motif, similar to the sequence found in S1 and S2. However, our data suggests that S1 and S2 are competing for the recognition site of a mRNA destabilizing protein. Downregulation of VEGF by DS-085 is mediated by triplex formation of the DNA strand, which inhibits the production of mRNA. We therefore see a proportional and direct relationship between the reduction in mRNA and the reduction of protein. If the mechanism of upregulation were mediated by increased mRNA production through inhibition of a repressor protein, we would see a similar, proportional increase between protein and mRNA. However, this is not the case for S1 and S2 where protein is increased by twofold compared to the control, while mRNA is only increased by 1.5 and 1.25 times respectively. Levels of mRNA are determined by the equilibrium that exists between synthesis and degradation therefore, an increase in stability will reduce degradation and cause an equilibrium shift resulting in higher levels of mRNA being present without an increase in synthesis. The improved mRNA stability, and hence the increased half-life, will result in a proportionally greater amount of protein produced per molecule of mRNA. In addition, stabilization/destabilization of mRNA has previously been shown to be the mechanism associated with increases on VEGF protein during periods of hypoxia (28) and has been well documented to play a role in regulation of other cellular elements such as transferrin receptors (29,30), elastin (31) and resistin (32).

To study the effects of the oligonucleotides on VEGF regulation in vivo; a rodent ocular model was chosen. VEGF isoforms are the same for all tissues and the eye makes an attractive organ to use, as the effects on ocular vascularization by changes in VEGF levels have been well described (review by (33)). In addition, the vasculature of the eye can be readily studied through opthalmologic examination. When introduced to the anterior chamber of the rat eye a strong neovascular response in the iris was observed for both S1 and S2. Likewise, sub retinal injection of S1 and S2 in both rats and mice resulted in a similar response in the retina in addition to the formation of micro aneurisms and leakage associated with the growth of new blood vessels. This pattern of neovascularization can also be observed in a rodent model with elevated expression of a VEGF transgene (34) in addition to patients suffering from diabetic retinopathy (35). Injection of S3 resulted in no observable response as was seen in the in vitro study. This provided a strong indication that the presence of S1 and S2 mediated an increase in the level VEGF protein with the effect of stimulating neovascularization. In addition, the sustained development and persistence of the new blood vessels was achieved by a single injection of the oligonucleotides, which makes it ideal in a gene therapy perspective. Comparisons of published sequences of the 5′-UTR show some variation in the sequence region proposed for the presence of a destabilizing element. However, as S1 and S2 both mediate a response in the mouse model, which lacks the S1 sequence, this provides evidence that inhibition is due to a shorter consensus sequence common to both S1 and S2. Responses to hypoxia are dependent on the presence of the hypoxia response element (HRM), which consists of a 6 base pair core consensus sequence (36). Similarly, low levels of glucose can mediate and increase in VEGF through the glucose response element (27). S1 and S2 both contain the element (T/A)GGGG which may represent the core recognition sequence of a destabilizing protein. Further examination of the human VEGF gene has revealed that several such elements exist in the 5′-UTR in addition to the 3′-UTR, which has also been identified as possessing destabilizing elements (9). The presence of a multiple number of destabilizing elements may serve as an effective means of regulating the rate of degradation i.e. the more sites that are occupied, the more rapid degradation becomes.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Shima, D. T., Kuroki, M., Deutsch, U., Ng, Y. S., Adamis, A. P., and D'Amore, P. A. (1996) J Biol Chem 271, 3877-3883.
2. Tischer, E., Mitchell, R., Hartman, T., Silva, M., Gospodarowicz, D., Fiddes, J. C., and Abraham, J. A. (1991) J Biol Chem 266, 11947-11954
3. van der Velden, A. W., and Thomas, A. A. (1999) Int J Biochem Cell Biol 31, 87-106.
4. Gulaniyogi, J., and Brewer, G. (2001) Gene 265, 11-23.
5. Miller, D. L., Dibbens, J. A., Damert, A., Risau, W., Vadas, M. A., and Goodall, G. J. (1998) FEBS Lett 434, 417-420.
6. Akdri, G., Nabari, D., Finkelstein, Y., Le, S. Y., Elroy-Stein, O., and Levi, B. Z. (1998) Oncogene 17, 227-236.
7. Forsythe, J. A., Jiang, B. H., Iyer, N. V., Agani, F., Leung, S. W., Koos, R. D., and Semenza, G. L. (1996) Mol Cell Biol 16, 4604-4613.
8. Levy, A. P., Levy, N. S., and Goldberg, M. A. (1996) J Biol Chem 271, 25492-25497.
9. 9. Dibbens, J. A., Miller, D. L., Damert, A., Risau, W., Vadas, M. A., and Goodall, G. J. (1999) Mol Biol Cell 10, 907-919.
10. Im, S. A., Kim, J. S., Gomez-Manzano, C., Fueyo, J., Liu, T. J., Cho, M. S., Seong, C. M., Lee, S, N., Hong, Y. K., and Yung, W. K. (2001) Br J Cancer 84, 1252-1257.
11. Carrasquillo, K. G., Ricker, J. A., Rigas, I. K., Miller, J. W., Gragoudas, E. S., and Adamis, A. P. (2003) Invest Ophthalmol Vis Sci 44, 290-299
12. Krzystolik M. G., Afshari, M. A., Adamis, A. P., Gaudreault, J., Gragoudas, E. S., Michaud, N. A., Li, W., Connolly, E., O'Neill, C. A., and Miller, J. W. (2002) Arch Ophthalmol 120, 338-346.
13. Garrett, K. L., Shen, W. Y., and Rakoczy, P. E. (2001) J Gene Med 3, 373-383.
14. Alunni-Fabbroni, M., Manfioletti, G., Manzini, G., and Xodo, L. E. (1994) European Journal of Biochemistry 226, 831-839.
15. Baran, N., Lapidot, A., and Manor, H. (1991) Proceedings of the National Academy of Sciences of the United States of America 88, 507-511.
16. Dayn, A., Samadashwily, G. M., and Mirkin, S. M. (1992) Proceedings of the National Academy of Sciences of the United States of America 89, 11406-11410.
17. Krasilnikov, A. S., Panyutin, I. G., Samadashwily, G. M., Cox, R., Lazurkdn, Y. S., and Mirkin, S. M. (1997) Nucleic Acids Research 25, 1339-1346.
18. Samadashwily, G. M., and Mirkin, S. M. (1994) Gene 149, 127-136.
19. Arnold, R., Maueler, W., Bassili, G., Lutz, M., Burke, L., Epplen, T. I., and Renkawitz, R. (2000) Gene 253, 209-214.
20. Genuanio, R. R., and Perry, R. P. (1996) J Biol Chem 271, 4388-4395.
21. Spilsbury, K., Garrett, K. L., Shen, W. Y., Constable, I. J., and Rakoczy, P. E. (2000) American Journal of Pathology 157, 135-144.
22. Axel, D. I., Spyridopoulos, I., Riessen, R., Runge, H., Viebahn, R., and Karsch, K. R. (2000) J Vasc Res 37, 221-234; discussion 303-224.
23. Adamnis, A. P., Miller, J. W., Bernal, M. T., D'Amico, D. J., Folkrnan, J., Yeo, T. K., and Yeo, K. T. (1994) Am J Opthalmol 118, 445-450.
24. Aiello, L. P., Avery, R. L., Arrigg, P. G., Keyt, B. A., Jampel, H. D., Shah, S. T., Pasquale, L. R., Thieme, H., Iwamoto, M. A., Park, J. E., and al., e. (1994) N Engl J Med 331, 1480-1487.
25. Siemeister, G., Martiny-Baron, G., and Marrae, D. (1998) Cancer Melastasis Rev 17, 241-248.
26. Ding, I., Liu, W., Sun, J., Paoni, S. F., Hernady, E., Fenton, B. M., and Okunieff, P. (2003) Adv Exp Med Biol 530, 603-609.
27. lida, K., Kawakami, Y., Sone, H., Suzuki, H., Yatoh, S., Isobe, K., Takekoshi, K., and Yamada, N. U.-. (2002) Life Sciences 71, 1607-1614.
28. Shima, D. T., Deutsch, U., and D'Amore, P. A. (1995) FEBS Lett 370, 203-208.
29. Koeller, D. M., Horowitz, J. A., Casey, J. L., Klausner, R. D., and Harford, J. B. (1991) Proc Natl Acad Sci USA 88, 7778-7782.
30. Mullner, E. W., and Kuhn, L. C. (1988) Cell 53, 815-825.
31. Hew, Y., Lau, C., Grzelczak, Z., and Keeley, F. W. (2000) J Biol Chem 275, 24857-24864.
32. Kawashima, J., Tsuruzoe, K., Motoshima, H., Shirakarni, A., Sakai, K., Hirashima, Y., Toyonaga, T., and Araki, E. (2003) Diabetologia 46, 231-240.
33. Witmer, A. N., Vrensen, G. P., Van Noorden, C. J., and Schlingemann, R. O. (2003) Prog Retin Eye Res 22, 1-29.
34. Baffi, J., Bymes, G., Chan, C. C., and Csaky, K. G. (2000) Invest Opthalmol Vis Sci 41, 3582-3589.
35. Moore, J., Bagley, S., Ireland, G., McLeod, D., and Boulton, M. E. (1999) J Anat 194 (Pt 1), 89-100.
36. Su, H., Arakawa-Hoyt, J., and Kan, Y. W. (2002) Proc Natl Acad Sci USA 99, 9480-9485.

What is claimed is:

1. A pharmaceutical composition comprising a nucleic acid molecule and a pharmaceutically acceptable adjuvant, carrier or diluent, wherein the nucleic acid molecule consists essentially of at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

2. A composition according to claim 1, wherein each of Aₙ and Bₘ comprises the same or different nucleotides selected from A, T, U, G and C or derivatives or analogues thereof.

3. A composition according to claim 1, wherein the nucleic acid molecule is capable of enhancing the expression of VEGF in a host cell that expresses VEGF.

4. A composition according to claim 1, wherein the nucleic acid molecule is capable of enhancing the stability of a transcript from the VEGF gene in a host cell that expresses the transcript.

5. A composition according to claim 1, wherein the nucleic acid molecule comprises one or more tandem repeats of the nucleotide sequence represented by formula (I).

6. A composition according to claim 1, wherein the nucleic acid molecule is represented by the formula:
[AₙWGGGGBₘ]ₚ (II)
wherein:
Aₙ, Bₘ and W are as defined for formula (I); and
p is an integer from 2 to about 20.

7. A composition according to claim 1, wherein the nucleotide sequence is selected from any one of AGGGG [SEQ ID NO:1], TGGGG [SEQ ID NO:2] or UGGGG [SEQ ID NO:3].

8. A composition according to claim 6, wherein the nucleic acid molecule consists essentially of one or more sequences selected from GGAGGAGGGGGAGGAG [SEQ ID NO:4] or AGGAAGAGGAGAGGGG [SEQ ID NO:5].

9. A composition according to claim 1, wherein the nucleic acid molecule consists essentially of a nucleic acid sequence corresponding to an untranslated region of a VEGF transcript or portion thereof at least 12 to about 500 nucleotides in length.

10. A composition according to claim 1, wherein the nucleic acid sequence is selected from SEQ ID NO:6 and 7.

11. A composition according to claim 1, wherein the nucleic acid molecule comprises a sequence that displays at least 90% identity to a portion of a nucleic acid sequence selected from SEQ ID NO:6 and 7, and destabilizes a transcript to which it is operably connected to thereby increase the amount of VEGF in a host cell that expresses VEGF.

12. A composition according to claim 1, wherein the nucleic acid molecule is an oligonucleotide that comprises at least one sequence represented by the formula (I).

13. A composition according to claim 12, wherein the oligonucleotide comprises at least about 5 nucleotides.

14. A composition according to claim 12, wherein the oligonucleotide is selected from any one of SEQ ID NO: 9-36.

15. A composition according to claim 12, wherein the oligonucleotide is nuclease resistant.

16. A composition according to claim 1, wherein the nucleic acid molecule is a polynucleotide comprising a nucleotide sequence that encodes a transcript consisting essentially of at least one nucleotide sequence represented by the formula (I).

17. A composition according to claim 1, wherein the nucleic acid molecule is in a construct and is operably connected to a promoter.

18. Use of nucleic acid molecule in the manufacture of a medicament for treating, preventing or ameliorating the symptoms of a condition that benefits from enhanced angiogenesis or vascularization, wherein the nucleic acid molecule consists essentially of at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

19. A method for enhancing the expression of VEGF, comprising introducing a nucleic acid molecule into a host cell that expresses VEGF, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

20. A method for treating, preventing or ameliorating the symptoms of a condition that benefits from enhanced angiogenesis or vascularization, the method comprising contacting a tissue associated with the condition and in which VEGF is expressible with a nucleic acid molecule in an amount effective to increase the expression or amount of VEGF in the tissue, wherein the nucleic acid molecule comprises at least one nucleotide sequence represented by the formula:
AₙWGOOGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

21. A method according to claim 20, wherein the condition is an ischemic condition.

22. A method according to claim 20, wherein the ischemic condition is selected from the group consisting of cerebral ischemia; intestinal ischemia; spinal cord ischemia; cardiovascular ischemia; myocardial ischemia associated with myocardial infarction; myocardial ischemia associated with congestive heart failure (CHF), ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies.

23. A method for increasing angiogenesis or vascularization in a tissue in which VEGF is expressible, the method comprising contacting the tissue with a nucleic acid molecule according in an amount effective to increase the expression or amount of VEGF in the tissue, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

24. A method according to claim 23, wherein the tissue is selected from brain tissue, intestinal tissue, spinal tissue, myocardial tissue, ocular tissue, liver tissue, kidney tissue, skin tissue, penile tissue, tissue containing a wound or graft tissue.

25. A method for increasing angiogenesis or vascularization in a subject, the method comprising administering to the subject an effective amount of a nucleic acid molecule to thereby increase angiogenesis or vascularization, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

26. A method for preventing or treating an ischemic condition or for reducing, preventing or treating ischemia-related tissue damage in a subject, the method comprising administering to the subject an effective amount of a nucleic acid molecule to thereby treat the ischemic condition or to reduce the tissue damage, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

27. Use of a nucleic acid molecule in the manufacture of a medicament for increasing angiogenesis or vascularization, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

28. Use of a nucleic acid molecule in the manufacture of a medicament for preventing or treating an ischemic condition, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

29. Use of a nucleic acid molecule in the manufacture of a medicament for reducing, preventing or treating ischemia-related tissue damage, wherein the nucleic acid molecule comprises at least one nucleotide sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

30. A nucleic acid construct comprising a sequence that encodes a RNA destabilizing element and that is operably connected to a heterologous polynucleotide, wherein the RNA destabilizing element comprises at least one sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.

31. A method for decreasing the stability of a transcript expressed from a polynucleotide, the method comprising operably connecting a RNA destabilizing element to the polynucleotide, wherein the RNA destabilizing element comprises at least one sequence represented by the formula:
AₙWGGGGBₘ (I)
wherein:
W is A, T or U;
Aₙ is a sequence of n nucleotides, wherein n is from 0 to about 11 nucleotides and wherein the sequence Aₙ comprises the same or different nucleotides selected from any nucleotide; and
Bₘ is a sequence of m nucleotides wherein m is from 0 to about 11 nucleotides and wherein the sequence Bₘ comprises the same or different nucleotides selected from any nucleotide.


SEQUENCE LISTING (40 sequences)

SEQ ID NO: 1 - DNA, 5 bp, Artificial sequence
  VEGF RNA destabilizing element
agggg

SEQ ID NO: 2 - DNA, 5 bp, Artificial sequence
  VEGF RNA destabilizing element
tgggg

SEQ ID NO: 3 - RNA, 5 nt, Artificial sequence
  VEGF RNA destabilizing element
ugggg

SEQ ID NO: 4 - DNA, 16 bp, Artificial sequence
  Oligonucleotide S1 comprising a VEGF RNA destabilising element
ggaggagggggaggag

SEQ ID NO: 5 - DNA, 16 bp, Artificial sequence
  Oligonucleotide S2 comprising a VEGF RNA destabilising element
aggaagaggagagggg

SEQ ID NO: 6 - DNA, 1039 bp, Homo sapiens
cgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccat

SEQ ID NO: 7 - DNA, 1923 bp, Homo sapiens
gccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaaagcattttgtattaaagaatttaattctgatctc

SEQ ID NO: 8 - DNA, 16 bp, Artificial sequence
  Oligonucleotide S3
gggaggagccgcagcc

SEQ ID NO: 9 - DNA, 12 bp, Artificial sequence
  Olgonucleotide comprising mRNA destabilising element
ggcttggggcag

SEQ ID NO: 10 - DNA, 12 bp, Artificial sequence
  Olgonucleotide comprising mRNA destabilising element
ctgggggctagc

SEQ ID NO: 11 - DNA, 12 bp, Artificial sequence
  Olgonucleotide comprising mRNA destabilising element
ggcttggggaga

SEQ ID NO: 12 - DNA, 12 bp, Artificial sequence
  Olgonucleotide comprising mRNA destabilising element
tgcttttggggg

SEQ ID NO: 13 - DNA, 13 bp, Artificial sequence
  Olgonucleotide comprising mRNA destabilising element
gggcaggggccgg

SEQ ID NO: 14 - DNA, 13 bp, Artificial sequence
  Olgonucleotide comprising mRNA destabilising element
gggtggagggggt

SEQ ID NO: 15 - DNA, 13 bp, Artificial sequence
  Olgonucleotide comprising mRNA destabilising element
ggagggggaggag

SEQ ID NO: 16 - DNA, 13 bp, Artificial sequence
  Olgonucleotide comprising mRNA destabilising element
gaggagagggggc

SEQ ID NO: 17 - DNA, 14 bp, Artificial sequence
  Olgonucleotide comprising mRNA destabilising element
tgggaggggaatgt

SEQ ID NO: 18 - DNA, 14 bp, Artificial sequence
  Olgonucleotide comprising mRNA destabilising element
gggcatgggggcaa

SEQ ID NO: 19 - DNA, 14 bp, Artificial sequence
  Olgonucleotide comprising mRNA destabilising element
aggagtttggggag

SEQ ID NO: 20 - DNA, 14 bp, Artificial sequence
  Olgonucleotide comprising mRNA destabilising element
tggtggggccaggg

SEQ ID NO: 21 - DNA, 15 bp, Artificial sequence
  Olgonucleotide comprising mRNA destabilising element
tggggagcttcagga

SEQ ID NO: 22 - DNA, 15 bp, Artificial sequence
  Olgonucleotide comprising mRNA destabilising element
gctttggggattccc

SEQ ID NO: 23 - DNA, 15 bp, Artificial sequence
  Olgonucleotide comprising mRNA destabilising element
tcgcccccaggggca

SEQ ID NO: 24 - DNA, 15 bp, Artificial sequence
  Olgonucleotide comprising mRNA destabilising element
aattgtggggaaaag

SEQ ID NO: 25 - DNA, 16 bp, Artificial sequence
  Olgonucleotide comprising mRNA destabilising element
cggaggcttggggcag

SEQ ID NO: 26 - DNA, 16 bp, Artificial sequence
  Olgonucleotide comprising mRNA destabilising element
gctgggggctagcacc

SEQ ID NO: 27 - DNA, 16 bp, Artificial sequence
  Olgonucleotide comprising mRNA destabilising element
cgacggcttggggaga

SEQ ID NO: 28 - DNA, 16 bp, Artificial sequence
  Olgonucleotide comprising mRNA destabilising element
acaggggcaaagtgag

SEQ ID NO: 29 - DNA, 17 bp, Artificial sequence
  Olgonucleotide comprising mRNA destabilising element
gcttttgggggtgaccg

SEQ ID NO: 30 - DNA, 17 bp, Artificial sequence
  Olgonucleotide comprising mRNA destabilising element
agccgcgggcaggggcc

SEQ ID NO: 31 - DNA, 17 bp, Artificial sequence
  Olgonucleotide comprising mRNA destabilising element
ggggtggagggggtcgg

SEQ ID NO: 32 - DNA, 17 bp, Artificial sequence
  Olgonucleotide comprising mRNA destabilising element
aggagggggaggaggaa

SEQ ID NO: 33 - DNA, 18 bp, Artificial sequence
  Olgonucleotide comprising mRNA destabilising element
agagggggccgcagtggc

SEQ ID NO: 34 - DNA, 18 bp, Artificial sequence
  Olgonucleotide comprising mRNA destabilising element
cttgagttgggaggggaa

SEQ ID NO: 35 - DNA, 18 bp, Artificial sequence
  Olgonucleotide comprising mRNA destabilising element
ttggtggggccagggtcc

SEQ ID NO: 36 - DNA, 18 bp, Artificial sequence
  Olgonucleotide comprising mRNA destabilising element
gcatgggggcaaatatga

SEQ ID NO: 37 - DNA, 20 bp, Artificial sequence
  Oligonucleotide
catcacgaagtggtgaagtt

SEQ ID NO: 38 - DNA, 20 bp, Artificial sequence
  Oligonucleotide
aacgctccaggacttatacc

SEQ ID NO: 39 - DNA, 17 bp, Artificial sequence
  Oligonucleotide
aggcaccagggcgtgat

SEQ ID NO: 40 - DNA, 20 bp, Artificial sequence
  Oligonucleotide
ttaatgtcacgcacgatttc